SEQUENCE LISTING (29 sequences)

SEQ ID NO: 1 - DNA, 59 bp, Human immunodeficiency virus type 1
gggtctctctggttagaccagatttgagcctgggagctctctggctaactagggaaccc

SEQ ID NO: 2 - DNA, 106 bp, Human immunodeficiency virus type 1
ctgggtctctctggttagaccagatctgagcctgggagctctctggctaactagggaacccactgcttaagcctcaataaagcttgccttgagtgcttcaagtagt

SEQ ID NO: 3 - DNA, 106 bp, Human immunodeficiency virus type 1; identical to SEQ ID NO: 2

SEQ ID NO: 4 - DNA, 106 bp, Human immunodeficiency virus type 1
ctgggtctctcttgatagaccaggtcgagcccgggagctctctggctagcaagggaacccactgcttaaagcctcaataaagcttgccttgagtgcttaaagtggt

SEQ ID NO: 5 - DNA, 105 bp, Human immunodeficiency virus type 1
ctgggtctctcttgatagaccaggtcgagcccgggagctctctggctagcaagggaacccactgcttaagcctcaataaagcttgccttgagtgcttaaagtggt

SEQ ID NO: 6 - DNA, 106 bp, Human immunodeficiency virus type 1
ctgggtctctcttgttagaccaggtcgagcccgggagctctctggctagcaggggaacccactgcttaaagcctcaataaagcttgccttgagtgcttaaagtggt

SEQ ID NO: 7 - DNA, 106 bp, Human immunodeficiency virus type 1
ctgggtctctcttgttagaccaggtcgagcccgggagctctctggctagcaagggaacccactgcttaaagcctcaataaagcttgccttgagtgcttaaagtggt

SEQ ID NO: 8 - DNA, 106 bp, Human immunodeficiency virus type 1
agaggtctctcttgttagaccaggtcgagcccgggagctctctggctagcaggggaacccactgcttaaagcctcaataaagcttgccttgagtgctaaaagtggt

SEQ ID NO: 9 - DNA, 106 bp, Human immunodeficiency virus type 1
ctgggtctctcttgttagaccaggtcgagcccgggagctctctggctagcaggggaacccactgcttgaagcctcaatacagcttgccttgagtgcttagagcggt

SEQ ID NO: 10 - DNA, 106 bp, Human immunodeficiency virus type 1
ctgggtctctctagttagaccagatctgagcctgggagctctctggttagctggagaacccactgcttaagcctcaataaagcttgccttgagtgctttaagtagt

SEQ ID NO: 11 - DNA, 106 bp, Human immunodeficiency virus type 1
ctgggtctctctaggtagaccagatcagagcctgggagctctctggctatctagggaacccactgcttaagcctcaataaagcttgccttgagtgctctgagcagt

SEQ ID NO: 12 - DNA, 21 bp, Artificial Sequence
  Description of Artificial Sequence Synthetic primer
tggttagaccagatctgagcc

SEQ ID NO: 13 - DNA, 21 bp, Artificial Sequence
  Description of Artificial Sequence Synthetic primer
tgactaaaagggtctgaggga

SEQ ID NO: 14 - DNA, 30 bp, Homo sapiens
tcccgtctagcacctttgatttctcccaaa

SEQ ID NO: 15 - DNA, 30 bp, Homo sapiens
tttgggagaaatcaaaggtgctagacggga

SEQ ID NO: 16 - DNA, 30 bp, Homo sapiens
tcccgtctagcacctttggcttctcccaaa

SEQ ID NO: 17 - DNA, 30 bp, Homo sapiens
tttgggagaagccaaaggtgctagacggga

SEQ ID NO: 18 - DNA, 40 bp, Human immunodeficiency virus type 1
gtactagcttgtagcaccatccaaaggtcagtggatatct

SEQ ID NO: 19 - DNA, 40 bp, Human immunodeficiency virus type 1
agatatccactgacctttggatggtgctacaagctagtac

SEQ ID NO: 20 - DNA, 40 bp, Human immunodeficiency virus type 1
ggtgtaacaagctggtgttctctcctttgttggcttcttc

SEQ ID NO: 21 - DNA, 40 bp, Human immunodeficiency virus type 1
gaagaagccaacaaaggagagaacaccagcttgttacacc

SEQ ID NO: 22 - DNA, 30 bp, Human immunodeficiency virus type 1
cccagcggaaagtcccttgtagcaagctcg

SEQ ID NO: 23 - DNA, 30 bp, Human immunodeficiency virus type 1
cgagcttgctacaagggactttccgctggg

SEQ ID NO: 24 - DNA, 40 bp, Human immunodeficiency virus type 1
tcagatctggtctaaccagagagacccagtacaggcaaaa

SEQ ID NO: 25 - DNA, 40 bp, Human immunodeficiency virus type 1
ttttgcctgtactgggtctctctggttagaccagatctga

SEQ ID NO: 26 - DNA, 40 bp, Human immunodeficiency virus type 1
tagccagagagctcccaggctcagatctggtctaaccaga

SEQ ID NO: 27 - DNA, 40 bp, Human immunodeficiency virus type 1
tctggttagaccagatctgagcctgggagctctctggcta

SEQ ID NO: 28 - DNA, 40 bp, Human immunodeficiency virus type 1
taagcagtgggttccctagttagccagagagctcccaggc

SEQ ID NO: 29 - DNA, 40 bp, Human immunodeficiency virus type 1
gcctgggagctctctggctaactagggaacccactgctta

(19) United States
(12) Patent Application Publication
(10) Pub. No.: US 2008/0193563 A1
(43) Pub. Date: Aug. 14, 2008

(54) HARNESSING WNT SIGNALING TO RESTRICT HIV REPLICATION

(75) Inventor: Lena Stern, Chicago, IL (US)

Correspondence Address: BARNES & THORNBURG LLP, P.O. BOX 2786, CHICAGO, IL, 60690-2786, US

(73) Assignee: RUSH UNIVERSITY MEDICAL CENTER, Chicago, IL (US)

(21) Appl. No.: 11/832,582

(22) Filed: Aug. 1, 2007

Related U.S. Application Data
(60) Provisional application No. 60/821,057, filed on Aug. 1, 2006.

(51) Int. Cl. A61K 33/14; A61P 31/18

(52) U.S. Cl. 424/677

(57) ABSTRACT

Activation of the Wnt signaling pathway potently restricts HIV replication both in peripheral blood mononuclear cells (PBMCs) and astrocytes. Inducing Wnt signaling, leading to the activation of β-catenin, for example, by the addition of lithium chloride (LiCl) inhibited the replication of a number of HIV isolates in PBMCs. Activating Wnt signaling, either with LiCl or other agents that target this pathway, is an approach to inhibit HIV replication.

This application claims priority to copending U.S. Ser. No. 60/821,057 filed Aug. 1, 2006.

The present disclosure shows that 1) IFNγ turns off the Wnt pathway in astrocytes and allows HIV to replicate in these cells that otherwise resist HIV replication; 2) turning on Wnt in PBMCs (white blood cells) leads to HIV inhibition, turning Wnt off leads to more HIV replication. These two pieces of information indicate that Wnt represses HIV in PBMCs. A Wnt activator, lithium, inhibited HIV replication in PBMCs as well as in a latent model of HIV.

### BACKGROUND

There are clinical and pathological manifestations of human immunodeficiency virus (HIV) infection in the central nervous system (CNS). The CNS is second only to the lung as the most commonly affected organ in acquired immune deficiency syndrome (AIDS) patients. Post-mortem studies, using in situ hybridization and immunohistochemistry, demonstrate HIV invasion of the CNS. This invasion is associated with a number of neurological disorders within a subset of HIV positive patients, the most severe of which is HIV-associated dementia (HAD) or encephalitis. While advances in antiretroviral therapy have reduced the incidence of severe neurologic disease among infected patients in the developed world, HIV-associated neurologic complications are a major problem in underdeveloped countries and may once again increase in the developed world as patients live longer and as more severe forms of HIV encephalitis are emerging in association with drug resistance. Additionally, more precise evaluation of neurocognitive impairment of HIV positive patients has revealed a prevalence (approximately 20%) of neurocognitive impairment despite highly active anti-retroviral therapy (HAART). This may not be surprising given that not all antiretroviral drugs penetrate the CNS equivalently and that some drugs may not penetrate at all.

HIV replication occurs in astrocytes. Astrocytes constitute 40-70% of cells in the human brain. They play pivotal homeostatic and regulatory functions in maintaining the integrity of blood brain barrier (BBB) and survival of neurons. A critical role of astrocytes is the regulation of extracellular levels of glutamate, which is a key excitatory neurotransmitter in the brain, but in high concentrations induces neuronal apoptosis. Dysregulation in astrocytes leads to a number of sequalae that impact a wide spectrum of neurologic diseases, including Parkinson's, Alzheimer, and HIV-associated dementia or cognitive-motor impairment. These alterations include dysregulated cytokine profile, leading to production of cytokines (TNFγ, IL-1β, TGF-β associated with neuronal apoptosis and/or an inability to scavenge for neurotoxins. Using in situ PCR or LCM, HIV DNA is detected within astrocytes of post-mortem brains of adult HIV positive patients, suggesting that astrocytes may be a major target for HIV infection. However, in vitro HIV infection of astrocytes is restricted, with production of few viral progeny. This restriction is unique because it leads to an initial burst of low-level HIV, followed by the accumulation of multiply spliced mRNAs (Tat, Rev, Nef) with the translation of these proteins on occasions, but without completing the viral life cycle. Productive infection can be re-established following stimulation with TNFγ and IL-1β but HIV levels still consistently remain far less than that documented in more HIV permissive cells, such as microglia, CD4+ T cells, and monocytes. Restricted HIV replication in astrocytes is beyond virus entry, as even cell lines transfected with CD4 and key chemokine co-receptors are limited in supporting HIV replication demonstrating defects in viral nuclear transport and poor translation of viral mRNAs into structural proteins. HIV pseudotyped with envelope glycoproteins of amphotropic murine leukemia virus or vesicular stomatitis virus, bypassing entry requirements, had similar viral kinetics with initial burst of virions, albeit to higher levels than wild-type HIV, followed by a persistent latent state. Astrocytes utilize alternate receptors for HIV entry, such as the human mannose receptor and D6. The mechanism(s) restricting HIV replication in astrocytes is still not clearly delineated.

Intracellular innate factors restrict retrovirus replication. A number of intrinsically expressed host proteins are linked to inhibiting retrovirus replication. The most notable of which are Apolipoprotein B mRNA-editing enzyme catalytic polypeptide-like-3G (APOBEC3G), which inhibits reverse transcription, and tripartrite interaction motif (TRIM) 51FN8, which is expressed in rhesus macaque cells and inhibits HIV-1 infection by targeting the HIV capsid. However, none of these innate HIV restrictive factors have been documented within resident brain cells.

Wnt-signaling in brain impacts HIV replication. TCF-4, a down stream transcriptional factor of the Wnt signaling pathway, is a repressor of HIV replication in astrocytes. Wnt genes are a large family that encode soluble secreted glycoproteins that regulate development during embryogenesis. They were first described in Drosophila but are highly conserved among invertebrate and vertebrae species. The term Wnt is an amalgam of wingless (Wg) and int, reflecting the origin of these genes in Drosophila and mice, respectively. Mutations in Wnt across species leads to a number of developmental defects. Wnt proteins are differentially expressed and regulate neurogenesis of the developing brain. There are reports that Wnt signaling regulates hippocampal neurogenesis in the adult brain. Blocking Wnt signaling in the adult brain abolished neurogenesis in vivo. Wnt signaling, in adults, may be manipulated to favor neurogenesis, which may be beneficial for Alzheimer's, Parkinson's, and HIV-associated encephalitis.

Wnt are a family of 19 soluble secreted glycoproteins that are involved in ubiquitous signal transduction pathways that regulate the transcriptional activity of hundreds of genes that impact cell differentiation, communication, apoptosis/survival and proliferation. Wnt signaling is initiated by binding of Wnt protein to the seven transmembrane Frizzled family of receptors.

A schematic illustration of both β-catenin-dependant and independent Wnt pathways is illustrated in FIG. 1. The canonical β-catenin dependent pathways of Wnt signaling is initiated by binding of Wnt proteins to one of the eight members of the Frizzled receptors. This signal transduction ultimately leads to stabilization of β-catenin because it is not phosphorylated by the serionine/theronine kinase (GSK-3). Active β-catenin binds a lymphoid enhancer binding factor (LEF)/T cell factor (TCF) family of transcription factors (LEF1, TCF-1, TCF-3, and TCF-4), displacing their repressors such as Groucho and this TCF: β-catenin complex translocates to the nucleus where it binds to TCF/LEF cognate DNA sequences, regulating gene transcription. TCF:β-catenin target genes include c-myc, cyclinD, TCF-4, LEF-1, c-Jun, CD44, among others. In the absence of a Wnt signal, defined by lack of Wnt protein binding to Frizzled, β-catenin is phosphorylated and associates with a protein complex (GSK, axin, and the tumor-suppressor protein denomatous polyposis coli; APC) that tags β-catenin for degradation. Without active β-catenin in the nucleus, TCF/LEF remains bound to their repressors and inhibits gene transcription.

Activation of Wnt signaling leads to TCF/LEF activation independent of β-catenin. Most prominent of these non-canonical/β-catenin independent pathways is through activation of calcium/calmodulin-dependent kinase II (camKII) and protein kinase C (PKC 2). Astrocytes can increase their intracellular calcium levels in response to glutamate, cytokines, or even incubation with cerebral spinal fluid (CSF) from HIV+ patients. Therefore, the in vivo microenvironment of astrocytes may regulate the activation of the canonical or non-canonical Wnt signaling pathways. Wnt signaling was believed to be diminished in mature blood cells (lymphocytes).

### SUMMARY

Methods and compositions to reduce HIV replication in target cells are disclosed. Methods to reduce HIV replication in target cells include activating an intrinsic signaling pathway to suppress HIV replication in vivo. Activation or induction of an intrinsic pathway includes, for example, activating the Wnt signaling pathway in target cells by lithium chloride to inhibit HIV replication.

In blood cells (peripheral blood monuclear cells, PBMCs) activation of Wnt leads to HIV inhibition. This finding is novel because Wnt was thought to play a key role in T cell maturation but after maturation, its role was believed diminished. To the contrary, Wnt was induced by T cell activation and by lithium in PBMCs. Harnessing the ability to induce Wnt to suppress HIV is a novel strategy to use a host molecular mechanism to restrict HIV. Further, lithium and other compounds that will induce Wnt activity is novel and is cost effective to inhibit HIV.

Despite success of anti-retroviral therapy against HIV, viral mutations continue to diminish its efficacy, necessitating new approaches. HIV replication is repressed by activation of the Wnt/β-catenin pathway. Wnt/β-catenin signaling regulates approximately 500 genes involved in diverse cell functions. The Wnt/β-catenin pathway can be activated by lithium via repression of a negative regulator of the Wnt pathway, glycogen synthase kinase 3 (GSK-3). Lithium inhibits HIV replication in vitro. Loss and gain of function studies showed that lithium inhibits HIV replication, without affecting cell survival or proliferation, in a Wnt/β-catenin-dependent manner. Inhibition of endogenous levels of Wnt/β-catenin enhanced HIV replication.

Current anti-HIV therapy generally relies on drugs that interfere with the viral life cycle. However, inducing an intrinsic and endogenous signaling pathway through natural/innate host factors offer several advantages. For example, the pathway is intrinsic, well-worked out, and agents exist to regulate its activity. An intrinsic host approach that is manipulated to reduce HIV replication is disclosed herein.

A method of reducing HIV replication in target cells includes the steps of:
(a) selecting a signaling component in a Wnt signaling pathway;
(b) modulating the signaling component to activate Wnt signaling pathway in the target cells; and
(c) determining the reduction of HIV replication in the target cells.

Wnt signaling pathway is activated by lithium chloride (LiCl). The Wnt signaling component is selected from a group of kinases designated GSK-3β, β-catenin, and TCF-4. The target cells include peripheral blood mononuclear cells. The target cells also include astrocytes. The target cells may also include T-cells.

A method of reducing HIV replication in a target cell includes the steps of:
(a) selecting a signaling component in a Wnt signaling pathway;
(b) modulating the signaling component by administering an agent to the cell to activate the Wnt signaling pathway; and
(c) reducing the HIV replication in the target cell.

A method to reduce HIV replication in a host target cell includes the steps of:
(a) selecting a signaling component in an intrinsic signaling pathway; and
(b) modulating the signaling component to inhibit HIV replication in the host target cell.

### BRIEF DESCRIPTION OF THE DRAWINGS

FIG. 1 illustrates (A) the β-catenin-dependent Wnt-signaling pathway. This pathway is initiated by the binding of Wnt proteins to Frizzled (Fz) and LRP 5 or 6 (LRP 5/6) co-receptors. This event leads to the activation of Disheveled (Dsh), which inhibits the serionine/theronine kinase (GSK-3) that can no longer phosphorylate β-catenin. Dephosphorylated β-catenin is stabilized by its lack of association with a destruction complex {GSK-3, APC, Axin, and casein kinase 1 (CK-1)}. Subsequently, the dephosphorylated β-catenin binds to TCF/LEF transcriptional factors and co-activators (CBP and p300), displacing their repressor. This complex enters the nucleus where it binds to TCF DNA sequences and activates gene transcription. In the absence of a Wnt signal, Dsh is inactive and incapable of inhibiting GSK-3. Active GSK-3 phosphorylates β-catenin and allows for its association with the destruction complex that targets β-catenin for degradation, resulting in its inability to bind to TCF/LEF, which remains associated with their repressors. (B) β-catenin-independent Wnt signaling. At least three mechanisms of β-catenin-independent Wnt signaling have been described. Wnt binding to Fz and perhaps other co-receptors {Knypek (Kny) or Ror2} leads to down stream signals involving activation of calcium/calmodulin-dependent kinase II (CamKII) and protein kinase C (PKC). Fz may also activate heterotrimeric GTP-binding proteins (GTP BP), leading to activation of phospholipase C (PLC) and phosphodiesterase (PDE) or it may recruit Dsh, leading to activation of small GTP-binding proteins, such as Rho and Cdc42.

FIG. 2 presents the purity of human fetal astrocyte (HFA) cultures. Flow cytometry was performed to evaluate the purity of the HFA cultures. Cells were stained for an astrocytes' marker (GFAP, A, B), a microglia marker (CD68, A, C), an astrocytes' precursor marker (nestin, B), or a neuron marker (MAP2, C). Dot plot shown are representative of typical purity of HFA cultures used.

FIG. 3 shows cytokine receptor expression on astrocytes. U87MG (A) or HFA (B) were left untreated or treated with GM-CSF (50 ng/ml), IFNγ100 ng/ml) or TNFγ100 ng/ml). Cytokine concentration was based on the concentration determined to induce maximal HIV expression, except for TNFγ, which did not induce HIV. Percent expression of GM-CSF receptor (R), IFNγR, and TNFγR was measured 24 hours post-stimulation by flow cytometry. Data is representative of three experiments. Error bars represent standard deviations of the mean.

FIG. 4 demonstrates the effect of GM-CSF and IFNγ pre-stimulation on HIV replication in astrocytes. U87MG (A) and HFA (B) were pre-stimulated with increasing doses of either GM-CSF or IFNγ for 24 h followed by HIV-BAL infection for 24 hours. HIV replication was determined by measuring HIV p24 levels by ELISA seven days post-infection. The dotted line represents basal level of HIV infection of astrocytes without cytokine pre-treatment. Data is representative of three experiments performed in quadruplicate. Error bars represent standard deviations of the mean. Asterisks denote significant values (p<0.05) in comparison to untreated cultures as evaluated by analysis of variance.

FIG. 5 demonstrates the effects of GM-CSF and INFγ on the expression of receptors and chemokine co-receptors relevant to HIV replication. U87MG (A) and HFA (B) were left untreated or treated GM-CSF (50 ng/ml) or IFNγ (100 ng/ml) for 24 h. Expression of receptors on the x-axis was measured by flow cytometry. Isotype refers to mouse IgG. Data is representative of three experiments. Error bars represent standard deviation of the mean. (C). IFNγ up-regulates D6 on U87MG cells. U87MG were left untreated or treated with IFNγ. After 3 days, the cells were immunostained with anti-D6 or isotype antibodies. The first histogram (peak) represents isotype control; the dark shaded histogram represents untreated cultures, and the third histogram (peak) represents IFNγ-treated cultures. Data is typical of quadruplet experiments.

FIG. 6 shows a comparative analysis of early HIV transcription between untreated and infected IFNγ gamma treated astrocytes.

FIG. 7 is a description of a chromatin immunoprecipitation assay (ChiP). The assay involves 1) cross-linking proteins to DNA in vivo using formaldehyde, 2) lysing the cells and shearing the DNA by sonication to small fragments (300-500 bp), 3) immunoprecipitating (IP) the protein of interest bound to the DNA so that both protein and DNA will be immunocomplexed together; 4) disrupting the immunoprecipitated protein/DNA complex by either heating or Proteinase K digestion; and 5) using DNA primers to amplify the DNA sequence of interest. Signals of the appropriate size indicate association of the protein of interest with the DNA fragment amplified. A positive control consists of amplifying total DNA after step 2 (input control). An additional control is proceeding with step 3 (IP) without adding a specific antibody (no antibody control), which may yield low-level of input DNA.

FIG. 8 Chromatin structures of TAR and LTR sequences are competent for transcriptional activities. U87MG cells were HIV infected or left uninfected. At 96 h, ChIP was performed by immunoprecipitating DNA associated with acetylated histones by using acetylated histone H2B antibody or no antibody. The immunoprecipitated DNA was subsequently amplified for TAR sequences between genomic locations +1 and +153 (A) and LTR sequences between genomic locations −460 and −206 (B). Conventional ChIP assay controls were included and shown in this figure, such as an “input control,” referring to amplification of the DNA before the IP step, and a “no antibody control,” referring to amplification of DNA after the IP step but without the addition of an acetylated histone H2B antibody. Data are representative of at least three experiments. Ac, acetylated; Ab and ab, antibody; pos., positive; neg., negative.

FIG. 9. shows TCF-4 is immunoprecipitated with HIV TAR in untreated but not IFNγ-treated cultures. U87MG were left untreated or primed with IFNγ, infected with HIV, and ChiP performed using TCF-4 antibody for immunoprecipitation (IP). ChiP assay is described in detail in FIG. 7(A) comparison between untreated and IFNγ-treated TAR DNA immunoprecipitated with TCF-4; (b) additional controls of uninfected cultures, input DNA, and no antibody controls.

FIG. 10 (A) shows transfection efficiency of U87MG and HFA. The cells were transfected with GFP (pmaxGFP) plasmid using the neuclofector system (Amaxa). Expression of GFP was evaluated by flow-cytometry at day 3 post-transfection. The dark histogram represents an isotype control and the lighter histogram peak represents GFP expression. TCF-4 inhibition abrogates restriction to HIV replication in untreated/non-IFNγ-primed cells. U87MG (i) or primary human fetal astrocytes (ii) were transfected with TCF-4 dominant negative (DN) mutant or GFP construct, maintaining the DNA amount constant between the two cultures. The cells were then infected with HIV. Data represent mean HIV p24 (pg/ml)+/−SEM. HIV p24 was measured by conventional ELISA at day 7 post-infection. Asterisks in (i) and (ii) denote p=0.008 and p=0.002, respectively calculated using Wilcoxon rank-sum test between TCF-4 DN and GFP transfected cells.

FIG. 11 Impact of IFN-γ on Wnt signaling. U87MG cells were left untreated or treated with IFN-γ for 24 hrs and then transfected with either TOPflash (containing four native TCF/LEF binding sites) and Renilla constructs or GFP and Renilla constructs. Transfected cells were then cultured with or without their initial treatment (with or without IFN-γ). Luciferase relative light activity (relative light units) was measured 24 hrs later using a luminometer and normalized to Renilla activity. Data are based on at least three experiments and are presented as the increase (n-fold) in luciferase relative light units over Renilla ±standard deviation. The asterisk shows P<0.0001 between untreated (first column) and IFN-γ treated (second column) cultures using the Bonferroni multiple comparison test.

FIG. 12 presents identification of TCF-4 binding sites in HIV LTR. A genomic search was conducted as described herein. Site of TCF-4 binding in LTR is indicated.

FIG. 13 (A) shows that LiCl induces β-catenin expression. CD3+ T cells were isolated by negative immunoselection and left unstimulated or stimulated with anti-CD3/CD28 with or without LiCl (5 mM). Western blot was performed at 6 and 24 hrs for dephosphorylated (active) β-catenin or GAPDH expression. Positive control for active β-catenin is the A431 extract (US biological, Swampscott, Mass.). A minor band is visible in the original autoradiograph for unstimulated cells that becomes more visible after 6 hrs. (B) LiCl, a Wnt pathway activator, inhibits HIV replication. CD3+ T cells were treated with anti-CD3/CD28 and LiCl (5 mM) then infected with HIV Bal. Data represent mean p24 (pg/ml) expression at day seven +/−SD.

FIG. 14 shows that LiCl restricts replication of HIV-IIIB (X4).

FIG. 15 shows that LiCl restricts replication of HIV-Bal (R5).

FIGS. 16-19 demonstrate that LiCl inhibits a number of strains of HIV in PBMCs as indicated. PBMCs were treated with LiCl and infected with a number of primary HIV isolates. HIV replication was measured by p24 ELISA on day 7-post-infection.

FIG. 20 Active Wnt signaling in astrocytes. U87MG were transfected with either the TOPflash (containing four native TCF/LEF binding sites) and Renilla constructs or GFP and Renilla constructs. Luciferase relative light activity (RLU) was measured 24 hrs later using a luminometer and normalized to Renilla activity. Data is based on at least three experiments and is presented as fold increase in Luc RLU over Renilla +/−SD. Asterisk denote p<0.0001 between TOPflash and GFP transfected cultures using Bonferroni multiple comparison test.

FIG. 21 (A) Transfection efficiency of U87MG and HFA. The cells were transfected with GFP (pmaxGFP) plasmid using the neuclofector system (Amaxa). Expression of GFP was evaluated by flow-cytometry at day 3 post-transfection. The black histogram represents an isotype control and the gray histogram represents GFP expression. Transfection efficiency of U251MG was similar to U87MG. (B) Inhibition of canonical Wnt signaling induces HIV replication in astrocytes. U87MG (i), U251MG (ii) or primary human fetal astrocytes (iii) were transfected with a TCF-4 dominant negative (DN) mutant construct, a β-catenin DN mutant construct, or a GFP construct. Some cultures were treated as if they were transfected but no DNA was added (mock cultures). Total amount of DNA was constant between all transfected cultures. All cultures were then infected with HIV Bal; Data represent mean HIV p24 (pg/ml)+/−SEM. HIV p24 was measured by conventional ELISA at day 7 post-infection. Asterisks denote p<0.001 calculated using Wilcoxon rank-sum test between TCF-4 DN or β-catenin and GFP transfected cultures.

FIG. 22 TCF-4 is immunoprecipitated with HIV TAR-spanning region in untreated but not IFN_{J}-treated cultures. Untreated astrocytes are resistant to productive infection while IFN_{J}-treated astrocytes are susceptible to HIV infection. U87MG were left untreated or primed with IFN_{J}, infected with HIV, and ChIP performed using TCF-4 antibody for immunoprecipitation (IP). A comparison between untreated and IFN_{J}-treated TAR-spanning HIV DNA immunoprecipitated with TCF-4 is shown in A and the additional controls of uninfected cultures, input DNA, and no antibody controls are shown in B.

FIG. 23 LiCl induces β-catenin expression. PBMCs from healthy donors were left unstimulated or stimulated with α-CD3/CD28 with or without LiCl at 1 mM or 5 mM. Western blot was performed at 24 hrs for dephosphorylated (active) β-catenin or GAPDH expression. Positive control for active β-catenin is the A431 extract, which constitutively expresses β-catenin (US Biological, Swampscott, Mass.).

FIG. 24 Lithium induces Wnt signaling in PBMCs. PBMCs were stimulated with α-CD3/CD28 for 48 hrs then treated with LiCl (1 mM) for 24 hrs then transfected with either GFP, TOPflash, or FOPflash along with a Renilla construct. At 24 hrs post-transfection, luciferase activity was measured using the dual luciferase system and data presented as fold increase in luciferase (LUC) activity over Renilla activity. Data is representative of two independent experiments performed in triplicates. Untreated and lithium-treated TOPflash constructs, as determined by the Mann-Whitney test.

FIG. 25 Lithium inhibits HIV IIIB (A) and primary isolate (B) replication in PBMCs. PBMCs were stimulated with α-CD3/CD28, infected with 10 ng of HIV IIIB or 302151 and treated with 0-1 mM LiCl. HIV replication was determined by measuring p24 by ELISA on day seven. Data is representative of three experiments performed in triplicates. Asterisks denotes p<0.05.

FIG. 26 Impact of lithium on cell viability. PBMCs were stimulated with a CD3/CD28 for 24 hrs then treated with LiCl at dose indicated. Cell viability was measured by trypan blue exclusion assay (A) and annexin V/PI staining (B). Data represent triplicate experiments. In (C) PBMCs were isolated and left unstilumated or stimulated with α-CD3/CD28 for 48 hrs then loaded with CFSE tracking dye, and treated with 0, 1, 5 and 25 mM LiCl. Diluation of CFSE was evaluated by flow cytometry 96 hrs after lithium treatment. Data represent triplicate experiments.

FIG. 27 Lithium inhibits HIV through the Wnt/β-catenin pathway. PBMCs from healthy donors were stimulated with aCD3/CD28 for 48 h then transfected with a dominant negative (DN) construct of TCF-4, β-catenin, or green fluorescent protein (GFP) then infected with HIV IIIB (a) or primary strain 30215 (b). After 24 hrs, lithium at 1 mM was added to the cultures. HIV p24 ELISA was performed on day 7 post-infection Asterisks denotes p<0.01, as determined by the Dunnett Multiple Comparisons Test between cultures without the DN and those with the DN mutants, whether using GFP or Li as the comparative arm.

FIG. 28 Inhibiting basal Wnt activity enhances replication of HIV IIIB or primary 302151 stain. PBMCs were stimulated with a-CD3/CD28 for 48 hrs then transfected with GFP, Dominant negative (DN) TCF-4 or DN-β-catenin prior to infection with HIV IIIB (a) or 302151 (b). In c & d, PBMCs were stimulated with a-CD3/CD28, infected with HIV IIIB (c) or 302151 (d) for 2 hrs, washed extensively and treated with either 5 or 25 mM ALLN peptide or left untreated. HIV p24 levels were measured seven days post-infection or transfection. Asterisks denotes p<0.01 in comparison to no lithium treatment with TNFα, as determined by the Dunnett Multiple Comparisons Test.

FIG. 29 Lithium inhibits TNFa-mediated induction of HIV. J1.1 cells were treated with TNFa (100 U/ml) with or without LiCl (0-1000 mM) for three days and HIV replication monitored by p24 ELISA three days post-treatment. Basal level of HIV replication in J1.1 (without TNFa treatment) was <50 pg/ml. Data represent a minimum of three experiments. Asterisks denotes p<0.01 in comparison to no lithium treatment with TNFa, as determined by the Dunnett Multiple Comparisons Test.

FIG. 30 The structure of the transactivating region (TAR, framed) and downstream flanking area. Nucleotide positions are relative to the start of transcription in the position +1 nt. Sarch for putative TCF-4 binding sites within 1-153 nt TAR area revealed single site with 86% homology to TCF-4 consensus (framed). Single letter mismatches are shown with cyan; conservative positions in TAR highlighted with yellow. Alignment of several original HIV1 isolates and two lab strains pBa_L (AB221005) and HXB2 9K03455) revealed that TCF-4 putative site is very conservative, and only 4 HIV-1 isolates of 330 contained substitutions in this area.

FIG. 31 Comparative analysis of early and late HIV reverse transcription (RT) between infected untreated and infected IFN-γ treated astrocytes. U87MG cells were left untreated or treated with IFN-γ prior to HIV infection as described in Materials and Methods. Total DNA was isolated and amplified for tierh early HIV reverse transcription (reverse transcription initiation) using primer pair R and U5 at 24 h postinfection or late HIV transcription using primer pair R and 5NC at 96 h postinfection. (A) Comparison of amplified HIV DNA between untreated and IFN-γ-treated cultures. (B) All of the controls for real-time PCT including GAPDH amplification from HIV-positive (pos.) and -negative (neg.) cultures. Data shown are representative of at least two experiments. Rn stands for reading normalized, which equals the SyberGreen value divided by the ROX reference dye value.

### DETAILED DESCRIPTION

Currently there are approximately 40 million people world-wide infected with the human immunodeficiency virus (HIV). The majority of these individuals reside in resource poor settings, where the availability of highly active antiretroviral therapy (HAART) is limited. Even when available, the cost of HAART is often prohibitive and is associated with metabolic disorders. Cost limitation, drug toxicity, and the high mutation rate of HIV emphasize the need for continued efforts to identify new anti-HIV therapeutic agents. To identify new targets for HIV intervention, mechanism(s) by which certain cells resist HIV replication post-entry were sought. HIV replication is repressed in non-permissive cell targets by activation of the Wnt/β-catenin pathway. The Wnt/β-catenin pathway is activated by lithium. Lithium is a commonly used drug for the treatment of bipolar mood disorder. It has been in clinical use for over 50 year. Lithium inhibits the activity of both the α and β isoforms of Glycogen Synthase Kinase 3 (GSK-3), resulting in the activation of the Wnt signaling pathway. Lithium inhibits HIV in PBMCs through modulating the Wnt/β-catenin pathway. The Wnt/β-catenin pathway and lithium specifically suppress HIV in blood.

Evidence from astrocytes, which restrict HIV replication pre- and post-entry, suggest that TCF-4 represses basal and Tat-mediated transactivation of the HIV LTR in a β-catenin-independent manner. TCF-4 is a repressor of HIV replication, in PBMCs. Repression is β-catenin-dependant, as demonstrated by abrogation of the inhibitory effect when β-catenin activity was inhibited though its respective dominant negative mutant. Integral components of Wnt pathway, including β-catenin, are critical in repressing HIV replication in PBMCs.

Although antiretroviral drugs (ART) have successfully targeted components of the viral life cycle, such as fusion, reverse transcription, and viral protein processing, viral mutations against these targets continue to diminish its efficacy, necessitating new approaches. (1) Lithium inhibits HIV replication in PBMCs and (2) the Wnt/β-catenin pathway represses HIV in PBMCs and is a mechanism by which lithium exerts its anti-HIV effects. Lithium is a low-cost generic drug that, if developed for HIV treatment, will be considerably less expensive than current single or combined anti-HIV therapy. Side effects of lithium are well known and can be managed by frequent monitoring of lithium serum levels to avoid intoxication.

Lithium may inhibit HIV replication in lymphocytes by inducing the Wnt/β-catenin pathway leading to TCF-4 binding to an HIV TAR-spanning region and interfering with Tat-mediated transactivation. TCF-4 (a downstream effector of the Wnt/β-catenin pathway) is a reported repressor of HIV transcription. TCF-4 associates with HIV TAR-spanning region in infected cells that resist productive HIV infection. Lithium, through activation of the Wnt/β-catenin pathway, likely suppresses multiple strains of HIV in primary myeloid and lymphoid cell. Active Wnt/β-catenin signaling in astrocytes is associated with HIV repression in these cells, while up-regulating the Wnt/β-catenin pathway, through lithium treatment, inhibited HIV in lymphocytes.

Wnt is also critical in the regulation of thymocytes, specifically in the transition of thymocytes from the CD4-CD8-(DN) to the CD4+CD8+ (DP) stage at the pre-TCR checkpoint. Double knock out studies in mice of Wnt transcriptional factors (LEF/TCF) resulted in complete block of T cell development. Although Wnt signaling is prominent in hematopoiesis and especially in thymopoiesis, its activity was believed to be diminished in mature lymphocytes.

Several publications identify the downstream effector of the Wnt pathway, TCF-4, as a repressor of HIV transcription. A series of transfection experiments of an LTR-reporter construct along with TCF-4 and Tat expression vectors, reported that TCF-4 expression reduces both basal and Tat-mediated transactivation of the HIV LTR. HIV infection in cells that were transfected with dominant negative mutant constructs of TCF-4 or β-catenin were used, which would have inhibited the endogenous activity of both proteins, and demonstrated that inhibiting both molecules in the Wnt signaling pathway enhanced HIV replication. These data indicate that integral components of Wnt pathway, including β-catenin, are critical in repressing HIV replication. One report further suggested that TCF-4 and Tat physically bind, forming a stable complex detected in the cytoplasm and nucleus.

Lithium (Li⁺) is a commonly used drug for the treatment of bipolar mood disorder. It has been in clinical use since 1949 and FDA-approved for bipolar disorder since 1974. The pharmokinetics of lithium are well described and the recommended therapeutic plasma level is between 0.6-1.2 mM. The two major therapeutic targets of lithium are glycogen synthase kinase 3 (GSK-3) and signal transduction via inositol trisphosphate (IP₃). Lithium inhibits the activity of both the D and E isoforms of GSK3, resulting in the activation of the Wnt signaling pathway. At higher concentrations (4-5 mM) lithium inhibits inositol monophosphatase (IMPase) and inositol polyphosphatase, leading to a decreased IP₃ response. Reported site effects of continuous lithium use includes nodular growth and goiter in some patients. This effect may be explained by the in vitro findings that lithium at 4 mM significantly increases the proliferative capacity of thyrocytes. Lithium is teratogenic and thus is not recommended for use by pregnant women in their first trimester. The teratogenic affect may be due to lithium regulation of Wnt signaling, which is critical in fetal development. Lithium side effects vary depending on its concentration, with most patients at risk of toxicity if the plasma level exceeds 2 mM. At high concentrations, lithium can cause tremors and diarrhea, increase in urine volume, and reduction of renal concentration ability. Although the risk of lithium intoxication is a series side effect, it can be regulated by individual monitoring and dosage adjustments to avoid overdose, and excluding patients with kidney disease, dehydration, sodium deficiency, and interactions with other known drugs. Lithium continues to be a potent mood stabilizer with benefits that out weigh any mild-to-moderate side effects.

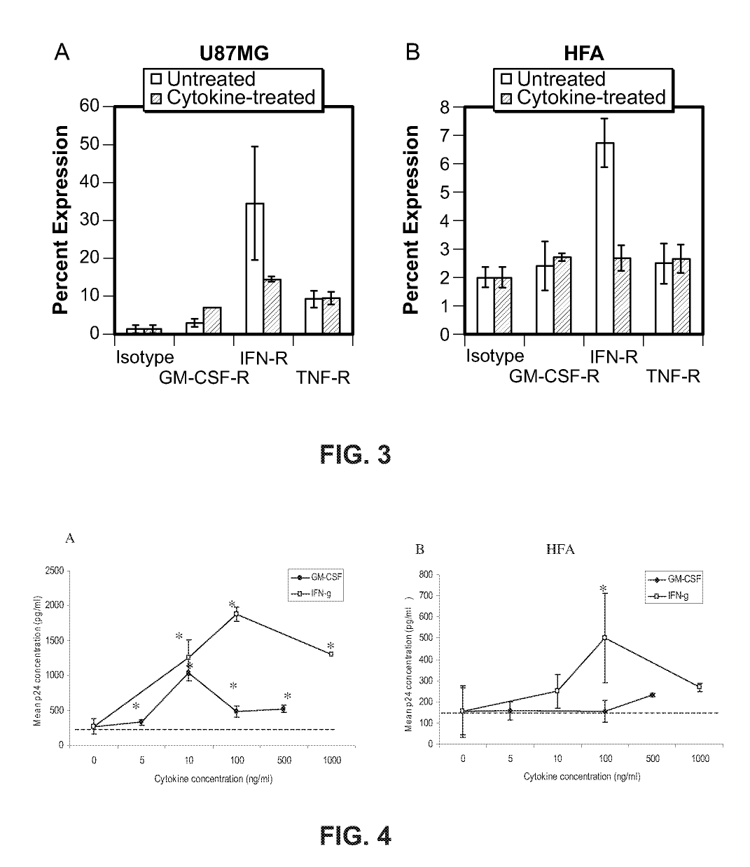

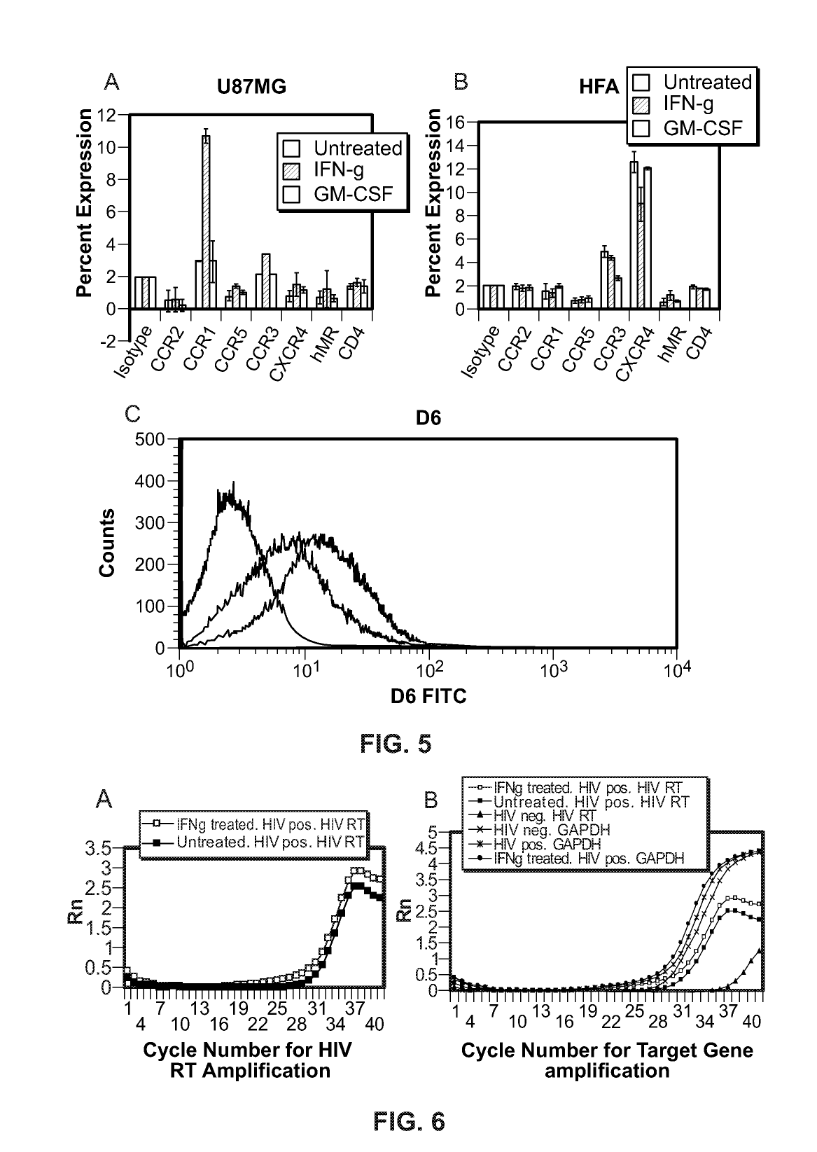

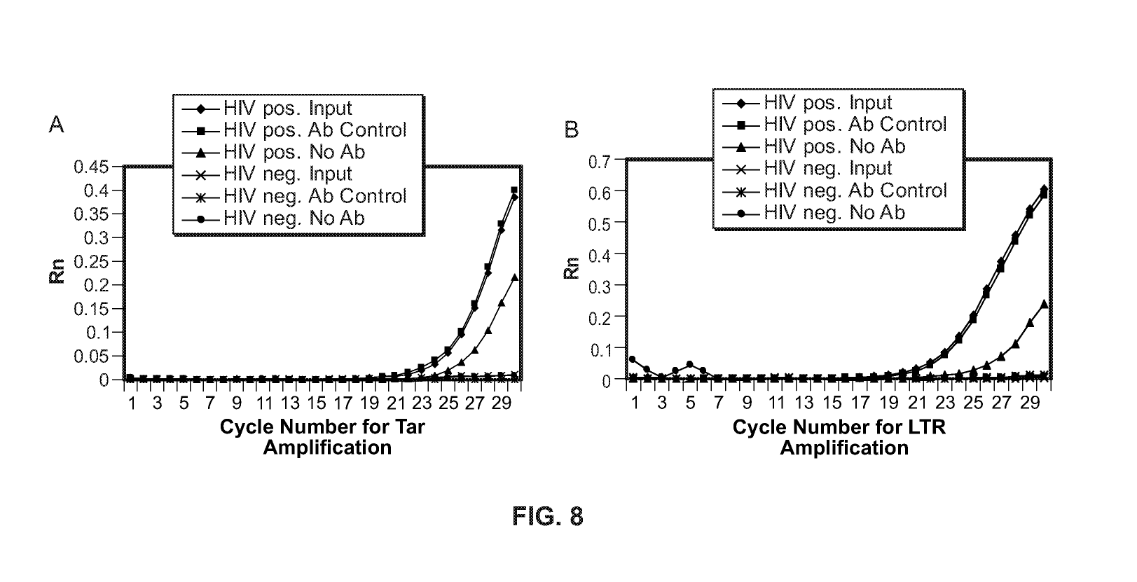

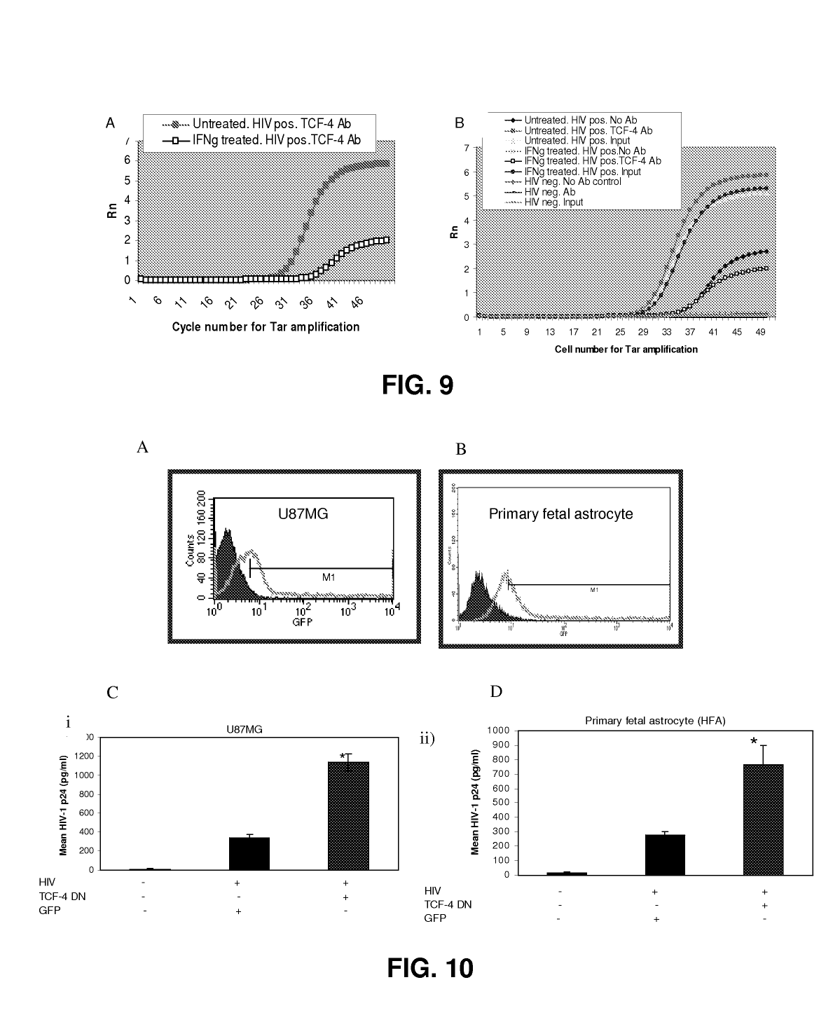

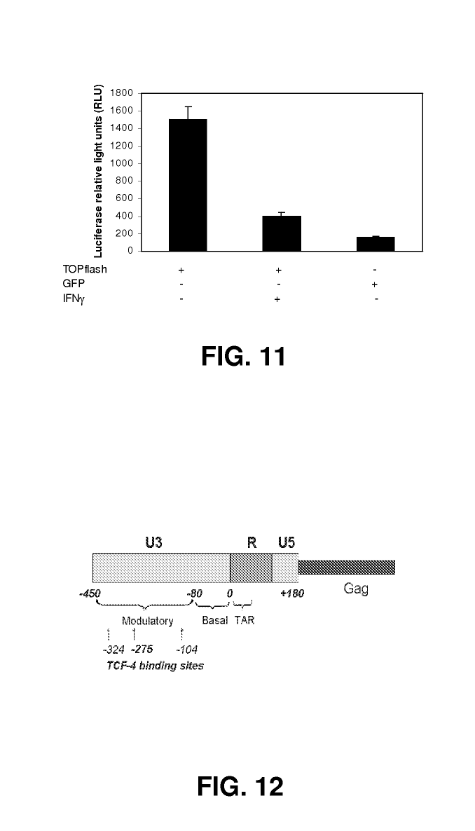

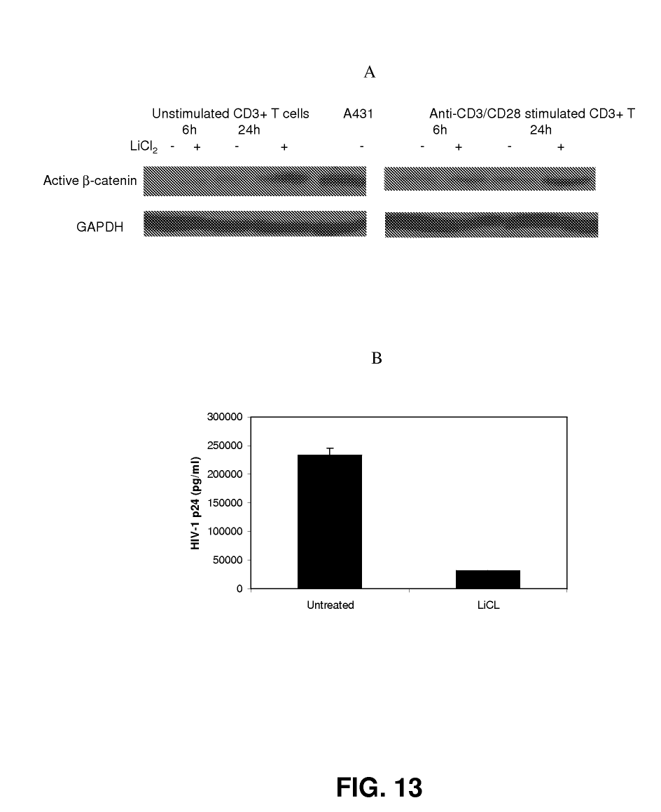

Human fetal astrocytes were purified as shown in FIG. 2. An IFNγ receptor is expressed on astrocytes and is down-regulated in response to IFNγ treatment (FIG. 3). IFNγ pre-treatment induces HIV replication in an astrocyte cell line and human primary fetal astrocytes (FIG. 4). The impact of IFNγ on HIV receptors and co-receptors (FIG. 5) is shown. Restriction to HIV replication in astrocytes and the ability of IFNγ to overcome this restriction is not likely at the level of HIV entry (FIG. 6) or chromatin modification (FIG. 8). Restriction to HIV replication in astrocytes and the ability of IFNγ to overcome this restriction is at the level of Wnt signaling as demonstrated by: a) an inverse relationship between TCF-4 association with HIV TAR region and induction of productive HIV replication in astrocytes (FIG. 9); b) Inhibition of TCF-4 activity, using a dominant negative mutant of TCF-4, abrogates restriction to HIV replication in untreated U87MG and primary fetal astrocytes (FIG. 10); c) Active Wnt signaling in astrocytes and its inhibition by IFNγ (FIG. 11); 6) Putative TCR-4 binding sites are identified on HIV LTR (FIG. 12 and Table I); and 7) A Wnt signaling activator (LiCl) inhibits HIV replication in primary human CD3+ T cells (FIG. 13).

### EXAMPLES

The following examples are for illustrative purposes and are not intended to limit the scope of this disclosure.

### Example 1

### Cytokine Priming of Astrocytes Augment HIV Replication

Cytokine (IL-7) pre-treatment of otherwise non-HIV permissive lymphocytes (naïve T cells) renders them permissive to HIV productive replication. Given that astrocytes in vitro do not support high level of HIV replication, key cytokines were evaluated to determine whether they can prime astrocytes to support HIV productive infection. The candidate cytokines, chosen on the basis of their relevance to HIV neuropathogenesis, were TNFγ, GM-CSF, and IFNγ. TNFγ is secreted by glial cells and linked to demyelination impaired glutamate scavenging by astrocytes and limited induction of HIV from astrocytes in culture. GM-CSF is also secreted by glial cells and correlates with elevated viral load in the cerebral spinal fluid (CSF) of HIV infected individuals and in mixed brain cell culture aggregates. IFNγ is secreted by lymphocytes that have infiltrated the CNS and by activated microglia and astrocytes, based on the rate model. IFNγ induces the expression of chemokine co-receptors on Simian astrocytes and HIV infection in microglia.

To evaluate the impact of TNFγ, GM-CSF, and IFNγ on modulating HIV replication in astrocytes, an astroglioma cell line (U87MG) and primary human fetal astrocytes (HFA) were used. HFA were purified from second trimester aborted fetuses, as described by Riveacia et al. (2005). A typical level of HFA purity is shown in FIG. 2 where the cultures were >95% positive for the astrocyte-specific marker glial fibrillary acidic protein (GFAP, FIG. 2A), less than 1% positive for the microglial marker, CD68 (FIG. 2A), 11% positive for nestin, a marker for precursor neural cells (FIG. 2B), and 4% positive for neurons as indicated by MAP2 immunostaining (FIG. 2C). Neurons do not support productive HIV replication and eventually die in these cultures. Microglia are extremely adherent and are depleted with the continuous passage of HFA, which was critical given that microglia replicate HIV efficiently.

Prior to evaluating if GM-CSF, IFNγ, and TNFγ can augment HIV replication of astrocytes, a question was whether U87MG and HFA express the respective cytokine receptors. U87MG and HFA were treated with or without the respective cytokine for 24 hours and cytokine receptor expression was evaluated by flow cytometry. Approximately 35% of U87MG expressed the IFNγ receptor and a low level of GM-CSF and TNFγ receptors (<8%; FIG. 3A). After 24-hour treatment of U87MG with IFNγ, IFNγ receptor expression was down-regulated by 2-fold to approximately 15% (FIG. 3B), while neither GM-CSF nor TNFγ treatments had an effect on their respective receptor expression (FIG. 3A). Untreated HFA expressed only low levels of IFNγ receptor (6%) and this expression was abrogated with IFNγ treatment. GM-CSF and TNFγ receptors were not expressed on HFA.

To determine if pre-stimulation of astrocytes primes astrocytes, for productive HIV infection, U87MG and HFA were pre-treated for 24 hours with TNFγ, GM-CSF, or IFNγ at various concentrations (0-1000 ng/ml) then infected with HIV-Bal at 10 ng of HIV p24/1×106 cells for 24 hours. The cells were then washed extensively or trypsinized to remove bound virus and propagated in the presence of the respective cytokine concentration. HIV infection was monitored by p24 ELISA seven days post-infection. Choice of duration of cytokine pre-treatment was based on time kinetics indicating that 24-hour pre-stimulation yielded the highest level of HIV infection. For U87MG, the effect of IFNγ appears to be dose dependent, with maximal induction of HIV replication at 10-fold higher than untreated/infected cultures. (FIG. 4A). GM-CSF pre-stimulation induced a 5-fold induction in HIV replication at 10 ng/ml but higher doses were not as effective (FIG. 4A). TNFγ pre-stimulation did not induce HIV replication. In HFA, IFNγ induced a 2-fold induction of HIV replication while GM-CSF (FIG. 4B) and TNFγ had no effect. Combining IFNγ, GM-CSF, and TNFγ to pre-stimulate U87MG and HFA prior to HIV infection had no additive or synergistic effects. Cytokine treatment post-infection did not induce HIV replication in U87MG and HFA. The finding that astrocytes, when primed by IFNγ, can be productively infected with HIV is significant because astrocytes make up approximately 50% of the CNS cell population while microglia make up only 10-20%. Therefore, even if the efficiency of infection in astrocytes is less than that of microglia, the total viral output from astrocytes could contribute significantly to the CNS viral load.

Because IFNγ was a potent cytokine in inducing HIV infection of astrocytes, it was determined whether IFNγ treatment modulated key HIV receptors CD4, human mannose receptor D6 and co-receptors (CCR1, CCR2, CCR3, CXCR4, and CCR5) for HIV infection. U87MG is human mannose receptor (hMR) negative (FIG. 5). However, hMR was not detected on HFA, which may be regulated by the differentiation stage of the HFA (FIG. 5 (B)). CD4 and hMR expression in both U87MG and HFA was not augmented by IFNγ or GM-CSF (FIG. 5(A)-(B)). CCR1 expression on U87MG was up-regulated by 4-fold post IFNγ while GM-CSF had no effect on its expression. HFA were positive for CXCR4 (12%), but its expression was not augmented by GM-CSF or IFNγ treatment (FIG. 5B). IFNγ was reported to enhance CXCR4 and CCR5 expression on simian adult astrocytes, which is in contrast to data on human fetal astrocytes highlighting a discrepancy between simian adult and human fetal astrocytes. Although IFNγ alone does not modulate CCR5 and CXCR4 expression, it can synergize with TNFγ to up-regulate these chemokine co-receptors in human fetal astrocytes. Infection of U87MG and HFA with a T-tropic isolate (IIIB), while it was productive, was not enhanced by cytokine pre-treatment. This may be a consequence of the laboratory adapted HIV IIIB, therefore primary T-tropic or dual tropic strains may still be responsive to IFNγ pre-treatment of astrocytes. Recently, the CC chemokine receptor D6 was identified as a functional co-receptor for primary HIV infection of astrocytes. D6, which upon binding to 13-chemokines, does not lead to a signal transduction but scavenges for those chemokines to reduce the inflammatory response. D6 is expressed on U87MG and IFNγ-up-regulated its expression by 6-fold (FIG. 5C). This data suggest that IFNγ-mediated up-regulation of D6 may be a mechanism to limit inflammation in the brain microenvironment.

### Example 2

### Restriction to HIV Replication and its Alleviation by IFNγ Priming

Restriction to HIV replication is not at the level of HIV entry and reverse transcription. To establish if the block to HIV replication in astrocytes is at the level of viral entry and whether IFNγ priming may overcome this restriction at the entry level, U87MG were left untreated or pre-treated with IFNγ for 24 hrs and then infected with HIV. Unbound virus was removed by trypsinization. Early HIV reverse transcription was evaluated by real-time PCR at 72 hrs post-infection by amplifying with the R/U5 primer pairs, which detect negative-strand “strong-stop” DNA indicative of reverse transcription initiation. The house keeping gene GAPDH was co-amplified as an internal control. Expression of early reverse transcription DNA was similar between cultures left untreated or treated with IFNγ (FIG. 6). Similar results were obtained for late HIV reverse transcription, amplified using primer pairs R/5NC, which amplify for late reverse transcripts containing positive-strand DNA after the second template switch beyond the primer binding site (PBS) and after 7 days of infection, representing more stable infection. This data indicates that: 1) The restriction to HIV replication is not at the level of entry, as HIV entered these cells and underwent reverse transcription, and 2) IFNγ priming does not enhance HIV entry or rate of reverse transcription in these cells. This data also indicate that restriction to HIV replication and IFNγ-induction of HIV replication is unlikely to be due at the level of virus entry and suggest that post-entry mechanisms may be prominent in this restriction and conversely the ability of IFNγ to overcome this restriction.

### Example 3

### Association HIV LTR and Acetylated Histones

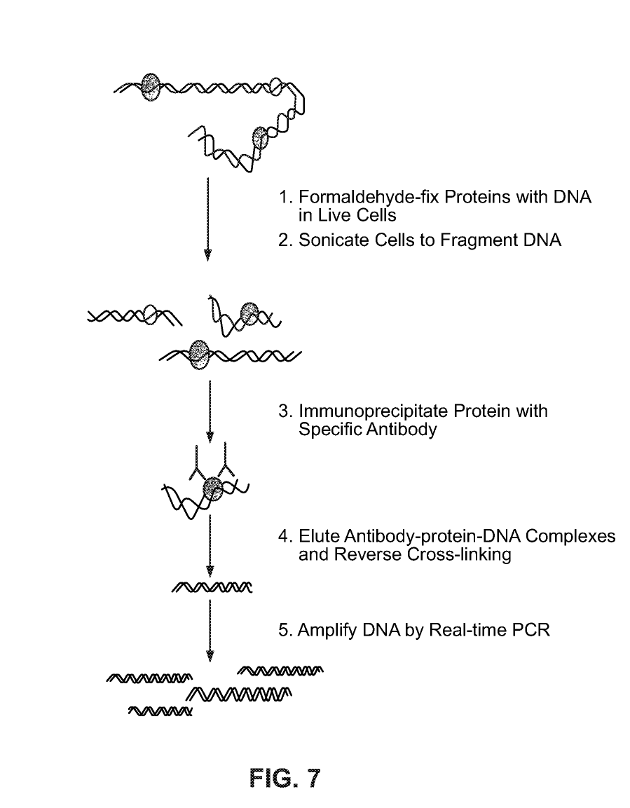

HIV LTR in untreated astrocytes is associated with acetylated histones indicative of regions of active gene transcription. To examine post-entry molecular pathways that may restrict HIV replication in astrocytes, the status of histone modification of the HIV LTR was evaluated. Several histones wrap around the DNA and when deacetylated, these substrates lead to chromatin condensation and are associated with inactive genes. However, after the histones, especially histone H1, are acetylated, the chromatin structure is modified becoming accessible to replication enzymes. Acetylated histones thus correlate with regions of active gene expression. Inhibition of HIV replication may be due to its association with deacetylated histones, leading to inactive gene transcription. To assess this possibility chromatin immunoprecipitation (ChIP) assay was performed on U87MG cells infected with HIV and DNA analyzed 72 hrs post-infection. The ChiP assay is described in FIG. 7. Unlike electrophoresis mobility shift assay (EMSA), ChiP allows for the in vivo determination of the association between specific DNA-binding proteins (e.g. histone/transcriptional factors) and specific region of the DNA. An antibody against acetylated histone H2B (Lys 5/12/15/20) was used to immunoprecipitate cellular DNA from HIV infected astrocytes. These complexes were then dissociated and the DNA amplified for HIV LTR region {genomic location between 2-256 and TAR region (genomic location 463-615)}. Detection of a signal indicates association with the acetylated protein. Input control refers to amplification of the DNA before the immunoprecipitation step and the no antibody control refers to amplification of DNA after the immunoprecipitation step but without the addition of a specific antibody. Both regions amplified for LTR and TAR were associated with acetylated histones (FIG. 8), as their respective DNA was amplified post-immunoprecipitation with the acetylated histone recognizing antibody. Low-level of HIV DNA was amplified after immunoprecipitation without using an antibody, reflecting the passage of some input DNA. This data indicate that the HIV promoter is associated with transcription-ready complexes. Thus, the block to restricted HIV replication in astrocytes is not caused by condensed chromatin association and subsequent inhibition of active HIV gene transcription.

### Example 4

### Immunoprecipitation of TCF-4 with HIV TAR

TCF-4 is immunoprecipitated with HIV TAR in astrocytes that restrict HIV replication but not in IFNγ-primed astrocytes, which support productive HIV replication. TCF-4, a Wnt signaling transcriptional factor, is defined as a transcriptional repressor of basal and Tat-mediated transactivation of the HIV LTR. To evaluate the role of TCF-4 restricted HIV replication in astrocytes and those primed with IFNγ which support productive HIV replication, ChiP was performed on U87MG left untreated or treated with IFNγ, infected with HIV, chromatin immunoprecipitated with a TCF-4-specific antibody, and DNA amplified for HIV TAR. TCF-4 was immunoprecipitated with TAR in untreated cultures but that this association is absent when the cells are primed with IFNγ (FIG. 9). Taken with the fact that untreated astrocytes do not support productive HIV replication, whereas IFNγ priming induces HIV replication (FIG. 4) suggest that there is an inverse relationship between TCF-4 association with TAR and HIV replication. These data point to a role of TCF-4 in regulating HIV replication in astrocytes.

### Example 5

### TCF-4 Inhibition Reverses the Restriction to HIV Replication

TCF-4 inhibition reverses the restriction to HIV replication in untreated/non-cytokine primed U87MG and human fetal astrocytes (HFA): Given the observed inverse relationship between HIV replication in astrocytes and TCF-4 and TAR association, whether via direct or indirect binding, the direct role of TCF-4 in HIV replication was evaluated by using a dominant negative mutant of TCF-4, which is mutated in its β-catenin binding sites and is a repressor of TCF-4 activity. U87MG and HFA were transfected with TCF-4 dominant negative (DN) mutant, or Green Fluorescence Protein (GFP) plasmid. Total amount of DNA remained constant between the cultures. Twenty four hours post-transfection, U87MG and HFA were infected with HIV and HIV replication was measured by p24 ELISA on day 7. Efficiency of transfection at day 3 post-transfection was at approximately 60% and 50% for U87MG and HFA, respectively, as measured by GFP expression (FIG. 10 A). Inhibiting TCF-4 in U87MG and HFA modulated HIV replication by 6- and 3-fold, respectively, in comparison to cells transfected with the GFP plasmid alone (FIG. 10B). This level of HIV replication post-TCF-4 dominant negative transfection of astrocytes is similar to that achieved by priming the cells with IFNγ. Priming the cells with IFNγ-prior to TCF-4 DN transfection did not demonstrate higher rate of HIV replication than IFNγ treated cultures alone. This data indicate that inhibition of TCF-4 activity removes the restriction to HIV replication in astrocytes and focuses on the Wnt pathway for targeted therapeutic intervention of intrinsic/naturally occurring mechanisms to restrict HIV replication.

### Example 6

### Active Wnt Signaling in Astrocytes and its Inhibition by IFNγ

Wnt signaling in astrocytes and its inhibition by IFNγ was investigated: Although inhibiting TCF-4 with a dominant negative mutant stresses the importance of active TCF-4 in restricting HIV replication, it is not informative regarding the mechanism by which IFNγ overcomes astrocyte restriction to productive HIV replication. To test whether astrocytes have active Wnt signal and that IFNγ impacts Wnt signaling pathway, U87MG were left untreated or IFNγ-treated then transfected with either a TCF-4 Luciferase construct (TOPflash) or GFP constructs and cultured with or without IFNγ. Luc activity was measured 24 hrs post-transfection TCF-4 reporter construct is an indicator of basal and inducible levels of Wnt signaling. Active Wnt signaling was detected in U87MG and IFNγ markedly reduced this signal by approximately 80% (FIG. 11). This data in conjunction with the TCF-4 ChiP and dominant negative data indicate that Wnt signaling is associated with HIV restriction in astrocytes and that IFNγ overcomes this restriction by reducing the potency of this pathway.

### Example 7

### Identification of Putative TCF-4 Binding Sites in the Promoter Region of HIV-1

TCF-4 binding sites were identified in the promoter region of HIV-1. Given that TCF-4 repress HIV replication, putative TCF-4 binding sites were sought in the promoter region of HIV-1 using information resources on the on-line database for eukaryotic transcription factors (TRANSFAC®, http://www.gene-regulation.com/pub/databases.html#transfac). Specifically, the “AliBaba2” program was used for predicting binding sites of transcription factor binding sites in an unknown DNA sequence. Additionally, for searching putative binding sites containing single-base substitutions, alignment tools in Vector NTI and OMIGA were aligned. TCF-4 has several names, such as immunoglobulin transcription factor 2 (ITF2), SEF2-1B, SEF2, and E2 2. TCF 4 binds to the specific sequence in the promoter region of various genes, but consensus sequence should include 5′-(A/T)(A/T)CAAAG-3′ stretch. This core binding sequence was found both in direct and reverse orientations depending on the gene. Using TRANSFAC database and the “AliBaba2” program, TCF-4 consensus in HIV-1 LTR region (GenBank accession number K03455). Initially the search was sought the first 1000 nucleotides from 5′-end, which includes LTR, TAR-binding site and the beginning of the gag gene (FIG. 12). All given positions are relative to the start of transcription, which was assigned position “0”. An exact match of the TCF-4 consensus was found at position 275 (FIG. 12 and Table 1). Additional searches for putative TCF-4 sites possibly including single substitution in the consensus yielded with −324 and −104 positions in the HIV-1 LTR. Because of LTR polymorphism among various strains, the search was performed for the regions in viral genome, which does not contain obvious TCF-4-binding-related sequences but are important for viral transcription. This search identified several possible TCF-4 binding sites in the HIV LTR (Table 1).

### Example 8

### A Wnt Pathway Activator (LiCl) Inhibits HIV Replication in Primary Human T Cells

LiCl inhibits the Wnt pathway by inhibiting GSK-30, leading to a dephosphorylated (active) β-catenin. To assess if Wnt pathway activation restricts HIV replication in targets other than astrocytes, the ability of LiCl to inhibit HIV replication in purified CD3+ T cells was evaluated. First, the impact of LiCl on β-catenin activation was evaluated in CD3+ T cells (FIG. 13A). CD3+ T cells were isolated by negative immunoselection (Miltenyi Biotech), treated with LiCl (5 mM), and left unstimulated or stimulated with anti-CD3/CD28. Expression of active β-catenin was evaluated by western blot at six and 24 hrs. The dose of LiCl used (5 mM) was the optimum dose for inducing β-catenin without significant cytotoxic effects, as evaluated by trypan blue exclusion and PI staining. LiCl induced active β-catenin as early as 6 hrs and more prominently by 24 hrs in both stimulated and unstimulated T cells. This data indicate that LiCl in T cells also activates β-catenin. To evaluate the impact of catenin activation on HIV replication in T cells, CD3+ T cells were isolated from healthy donors treated with anti-CD3/CD28 and LiCl (5 mM) for 24 hrs then infected with HIV Bal. LiCl potently inhibited HIV replication in T cells by approximately 8-fold (FIG. 13B). This data indicate that β-catenin-dependant Wnt signaling is associated with restricted HIV replication in T cells and that activation of this pathway is a viable approach to restrict HIV replication.

### Example 9

### Molecular Mechanism of HIV-Restricted Replication in Astrocytes

An innate molecular mechanism of HIV-restricted replication in astrocytes by the Wnt signal pathway is shown to restrict HIV replication in U87MG and primary fetal astrocytes. Wnt signaling is associated with HIV restriction and suggest that IFNγ overcomes this restriction by inhibiting this pathway. In support of extending the potential of this intrinsic anti-HIV molecular mechanism to other permissive targets activation of the Wnt pathway in primary T cells potently inhibits HIV replication. Wnt signaling is a powerful approach to limit HIV replication within and outside of the CNS.

Despite HIV pseudotyping or expression of key HIV receptors and co-receptors, HIV replication is restricted in astrocytes. This restriction is defined by a brief low-level burst of HIV replication followed by the accumulation of early/regulatory HIV mRNAs. Active Wnt signaling in astrocytes is an intrinsic molecular mechanism to restrict HIV replication. 1. TCF-4 is complexed with HIV TAR in astrocytes, as demonstrated by chromatin immunoprecipitation (FIG. 9). Transfecting astrocytes with a dominant negative mutant of TCF-4 abrogates the restriction for HIV replication in astrocytes (FIG. 10B). Basal Wnt signaling is active in astrocytes (FIG. 11).

Data using the dominant negative mutant of TCF-4 showing its ability to elude the restriction to HIV replication in astrocytes can be interpreted in one of two ways: 1) Given that the dominant negative TCF-4 is mutated in its β-catenin binding site indicates that the association between β-catenin and TCF-4 is critical for suppressing HIV replication and that repression of HIV replication occurs via the canonical/β-catenin-dependant Wnt signaling pathway; or 2) repression of HIV occurs through the non-canonical/β-catenin-independent pathway. β-catenin-independent pathways for Wnt signaling are well documented, the most prominent of which is the calcium/calmodulin pathway. Calcium is expressed in astrocytes and its levels are elevated in response to various stimuli, thus basal intracellular calcium levels in astrocytes may activate the Wnt signal pathway in a β-catenin independent manner, leading to activation of TCF and in turn its repression of HIV replication. These two alternative explanations converge on a final outcome of an association between an active Wnt signal, dependant or independent on β-catenin, and restriction of HIV replication in astrocytes. Active Wnt signaling in astrocytes is an intrinsic molecular mechanism to restrict HIV replication. Exploring components of this pathway in relation to restricted HIV replication in astrocytes allows for the design of specific strategies to manipulate these components in HIV permissive targets within and outside of the CNS to transfer this HIV restriction to them. Activation of this pathway to limit HIV replication in many cell types, for example, is accomplished by a suitable reagent such lithium chloride (LiCl) or by designing novel agents that activate this pathway. Based on the data and disclosure herein, new therapeutic strategies for limiting HIV replication are developed to inhibit HIV isolates that are resistant to current antiretroviral treatments.

### Example 10

### Active Wnt Signaling in Astrocytes is an Intrinsic Molecular Mechanism that Restricts HIV Replication

The status of Wnt signaling in astrocytes both basal and inducible Wnt activity can be examined in U87MG and HFA. These cells can be transfected with SuperTOPflash (SuperTOP) or SuperFOPflash (SuperFOP) luciferase reporter constructs. These plasmids were constructed by Dr. Randall Moon (University of Washington, Seattle, Wash.) and are about 100-fold more sensitive to Wnt signaling that the commercially available constructs used in FIG. 11. The constructs include eight native or mutated TCF/LEF binding sites cloned into the Mlu1 site of the pTA-Luc vector (Clontech) in SuperTOP and SuperFOP, respectively. The cells can also be co-transfected with pRL-TK plasmid (Renilla plasmid) as an internal control for transfection efficiency. These transfection experiments can also be conducted in the presence or absence of DICKKOPF-1 (DKK-1) inhibitor and ectopic axin, both down-regulate the pathway and provides a baseline of how active the pathway is in astrocytes. Additionally, qPCR on axin 2 is performed, which is often a direct target gene to confirm Wnt activation. To assess if astrocytes are Wnt responsive to ectopic ligands, SuperTOP- and SuperFOP-transfected astrocytes is treated with Wnt3a conditioned media.

For the canonical pathway, Wnt proteins bind to their respective receptors (Frizzled/LPR). For the canonical pathway to be active, astrocytes themselves have to secret Wnt proteins that function in an autocrine fashion. To determine if this is the case and identify which Wnt proteins are expressed, given that there at least 11 different Wnt proteins, an initial screening using a cDNA microarray-based gene expression profiling for Wnt gene families is performed. Specifically, RT-PCR for Wnt 2, 3, 4, 6, 11, 16, 5A, 7A, 14, 2B, 15 and GAPDH is performed using cDNAs from untreated U87MG and primary human fetal astrocytes using the SuperArray's MultiGene-12T RT-PCR profiling panel (SuperArrary Bioscience corporation, Frederick, Md.). Table 2 demonstrates the expected size of each product. To confirm expression of Wnt ligands from these pure astrocytes, media from U87MG and primary fetal astrocytes are be added to L cells, which are Wnt responsive and are stably transfected with TCF-4-reporter construct. Induction of reporter activity indicates that Wnt proteins are secreted by astrocytes. Utilizing the mRNA profile of Wnt mRNA generated by RT-PCR, dot blots from astrocyte conditioned media using Wnt-specific antibodies is performed to indicate if the detected Wnt mRNA specie is translated and released into the astrocyte media. Based on these experiments, commercially available antibodies is added to astrocytes transfected with SuperTOP and SuperFOP to determine if Wnt signaling is abrogated with neutralizing these secreted ligands. Neutralizing antibodies to Wnt proteins are available from a number of commercial vendors (R&D systems; Upstate, Santa Cruz). Recombinant Wnt proteins, Wnt3A or Wnt 5, is used as positive controls for the Wnt pathway. Identification of the Wnt proteins secreted by astrocytes and acting in autocrine fashion to activate the Wnt pathway are helpful in further investigating the relationship between Wnt signaling and restricted HIV replication in astrocytes in vitro, where specific Wnt proteins is blocked and HIV outcome assessed in astrocytes.

Given that dephosphorylation of β-catenin is a primary outcome of the canonical pathway, examine the status of β-catenin in astrocytes is examined, whether it is active (dephosphorylated form) or inactive (phosphorylated form). This is accomplished by performing Western blots for β-catenin using antibodies that recognize the dephosphorylated (active) and phosphorylated (inactive) forms of β-catenin (US biological, Swampscott, Mass. and Abcam Inc., Cambridge, Mass., respectively). Active Wnt signaling also leads to other downstream effects such as phosphorylation of Dishevelled or the LRP tails, both of which is assessed by Western blots using commercially available antibodies. Additionally, the downstream effects of activated β-catenin leads to its binding to TCF-4. This is evaluated by performing TCF-4 immunoprecipitation (IP) of nuclear extracts from U87MG and primary fetal astrocytes then blotting with anti-β-catenin antibody (active form). Controls of these IP and Western blot experiments includes IgG antibody for IP and performing these assays in parallel using the Wnt-responsive L cell line.

The Wnt pathway relies on calcium mobilization. Astrocytes contain intracellular calcium that is up-regulated in response to various stimuli, including binding of glutamate and epinephrine to their respective sites. This calcium induction leads to TCF-4 activation. To evaluate this non-β-catenin-dependent pathway, basal intracellular calcium levels in astrocytes are monitored using a fluorescence-based calcium detection assay (The FLIPR Calcium 3 Assay, Molecular devices, Sunnyvale, Calif.). Positive controls includes recombinant glutamate or epinephrine and negative control includes calcium chelators or inhibiting calcium-sensing receptors (CASRs) with NPS 89636. A parallel control for this assay includes peripheral blood mononuclear cells (PBMCs) stimulated with anti-CD3/CD28, which lead to enhancement of intracellular calcium in activated T cells. Further immunoprecipitation and Western blot of nuclear TCF-4 is evaluated with or without calcium chelators, such as EDTA. These chelators is added at concentrations that are not cytotoxic to the cells as evaluated by trypan blue exclusion assay and propidium iodide (PI) staining. These experiments is helpful to identify the components and type of Wnt pathway.

### Example 11

### Relationship Between Wnt Signaling and HIV Replication

The following are addressed: 1) whether knockdown/Wnt signaling inhibitor experiments abrogate the restriction to HIV replication in astrocytes; 2) whether β-catenin is associated with TCF-4 on the HIV LTR or whether TCF-4's associated with the HIV TAR independent of β-catenin, and 3) whether there are direct TCF-4 cognate binding sites on the HIV LTR and especially within the TAR region and what effects does mutation of these sites have on HIV transcriptional activation.

### Example 12

### Methods to Identify Components of Wnt Signaling that Impact HIV Replication

Experiments that target inhibition of Wnt signaling to assess its impact on HIV replication include treating the cells with: 1) siRNA to β-catenin; 2) antibodies or antagonist to Frizzled receptors (Wnt receptors), 2) Treating the cells with the cell permeable N-acetyl-Leu-Leu-norleucinal (ALLN) peptide which inhibits proteasome-mediated proteolysis and induces the accumulation of phosphorylated, ubiquitinated β-catenin; and 3) Antibodies to Wnt proteins secreted by astrocytes. All of these approaches are evaluated for their impact on cell viability and proliferation as determined by trypan blue exclusion assay, apoptosis (Annexin V/PI) assay, and Carboxyfluoresccin Diacctate Succinimidyl Ester (CFSE) dye tracking assay at various concentrations. Cells are treated as indicated herein, with the proper mock treated controls, infected with HIV, and HIV expression monitored over time using HIV p24 core ELISA. In some cases where the cells are treated with an inhibitor, this inhibitor is added pre-infection and kept post-infection as well. If an affect is observed, experiments are conducted to indicate if this affect is important at pre- or post-HIV entry. Experiments that target activating Wnt signaling include: 1) Transfection of active β-catenin cDNA 2) Transfection of a β-catenin construct encoding a dominant positive mutant of β-catenin 3) Treating the cells with LiCl which is a known activator of Wnt signaling through inhibiting GSK [symbol] (123), and 4) Inhibiting GSK3 using siRNA. Utilizing these three approaches generates a stronger line of experimentation, given that LiCl, while is an activator for Wnt pathway, may lack specificity for this pathway. Activation of calcium dependant pathway in astrocytes lead to use of inhibitors of calcium reagents such as EDTA that induce HIV replication in astrocytes. Therefore, calcium chelators are added to astrocytes prior to infection and maintained in the culture, HIV replication is monitored by p24 ELISA.

### Example 13

### Mechanism of TCF-4 Inhibition of HIV Basal and Tat-Mediated Transcription

It was reported that Tat binds to TCF-4 but the exact mechanism of TCF-4 inhibition of HIV basal and Tat-mediated transcription was not clear. Additionally, this study did not use whole virus but rather a series of gene-reporter transfection assays. Data using chromatin immunoprecipitation (ChIP) demonstrates that TCF-4 is immunoprecipitated with TAR region of HIV LTR (FIG. 9). 1) Tat binds to TCF-4 and this complex binds to TAR, altering the steoichemistry of Tat-TAR interaction leading to HIV inhibition or 2) A multi-complex is formed between TCF-4/β-catenin, Tat, and TAR that leads to HIV inhibition. To delineate this relationship as it is relevant to understanding how the end product of Wnt signaling (activation of TCF) may inhibit HIV replication, ChiP assays is performed on DNA from HIV infected astrocytes using three different antibodies to immunoprecipitate separately TCF-4,13-catenin (active form), and Tat, and then amplify for HIV TAR. TAR signal detection informs if there is a tri complex (Tat, β-catenin, TCF-4) or at least two hetrodimers without Tat association. Another possibility exists whereby, TCF-4 association with Tat leads to modulation of host proteins reported to be essential for the Tat-TAR interaction, specifically expression of Tat RNA Binding Proteins (TRBP), Cyclin T1, and CDK-9, identified by Western blotting to be part of the Tat-TAR complex

To establish the ability of TCF-4 to directly bind to sites within the HIV LTR, with special emphasis on the TAR region, electrophoresis mobility shift assay (EMSA) is performed. A putative TCF-4 binding sites in the HIV LTR is identified (FIG. 12 and Table 1). Positive control include a recombinant TCF-4 protein co-incubated with the synthesized probes. Specificity of any binding is determined by supershifting using a TCF-4 specific antibody compatible for EMSA. If these sites are needed to repress HIV replication, this is tested by knocking and/or mutating these sites from an HIV-LTR-reporter construct.

### Example 14

### Inactivation of Wnt Signaling by IFNγ

IFNγ may inactivate Wnt signaling in astrocytes leading to induction of HIV replication. This is based on:

1. TCF-4 is no longer immunoprecipitated with TAR region in IFNγ primed astrocytes (FIG. 10).

2. IFNγ treatment of astrocytes reduces their basal level of Wnt signaling by 80% (FIG. 11).

Therefore, it is likely that IFNγ signaling leads to the inhibition/inactivation of the β-catenin-dependent Wnt signaling pathway, resulting in the inability of TCF-4 to bind either directly or indirectly to HIV TAR and removing its repression on HIV replication. Astrocyte dysregulation correlates with the severity and the rate of HAD progression, highlighting a pivotal role for astrocytes in HIV neuropathogenesis.

Considerable examples exist where HIV manipulates the immune system to its favor, as in its ability to down-regulate CD4 and antigen presentation molecules (Histocompatibility (MHC) class I, II and CD1d. Wnt signaling through β-catenin is a survival signal as it is associated with up-regulation of the anti-apoptotic gene, Bcl-xL. IFNγ, conversely, is a pro-apoptotic signal for virally infected cells. Therefore, IFNγ-mediated inhibition of the Wnt pathway seems a logical consequence if the net effect of IFNγ is cell death rather than survival of the infected target. HIV may take advantage of this pathway by replicating in cells receiving an IFNγ signal that inhibits Wnt signaling. These studies are useful to gain an understanding of factors within the brain microenvironment that may influence higher HIV replication within astrocytes and consequently susceptibility to HIV-associated cognitive-motor impairment. Understanding the mechanism by which IFNγ can override astrocyte restriction to HIV replication may lead to novel therapies that target this mechanism.

The molecular mechanism by which IFNγ overcomes astrocyte restriction to HIV replication was investigated by: 1) Determining the impact of IFNγ on the Wnt signaling pathway; 2) Examining the link between these IFNγ affected molecules and their association with induction of productive HIV replication in vitro, and 3) Establishing the association between IFNγ in brain, viral load in brain, HIV replication in astrocytes, and severity of cognitive-motor impairment through in situ evaluation of adult human brain tissue and CSF.

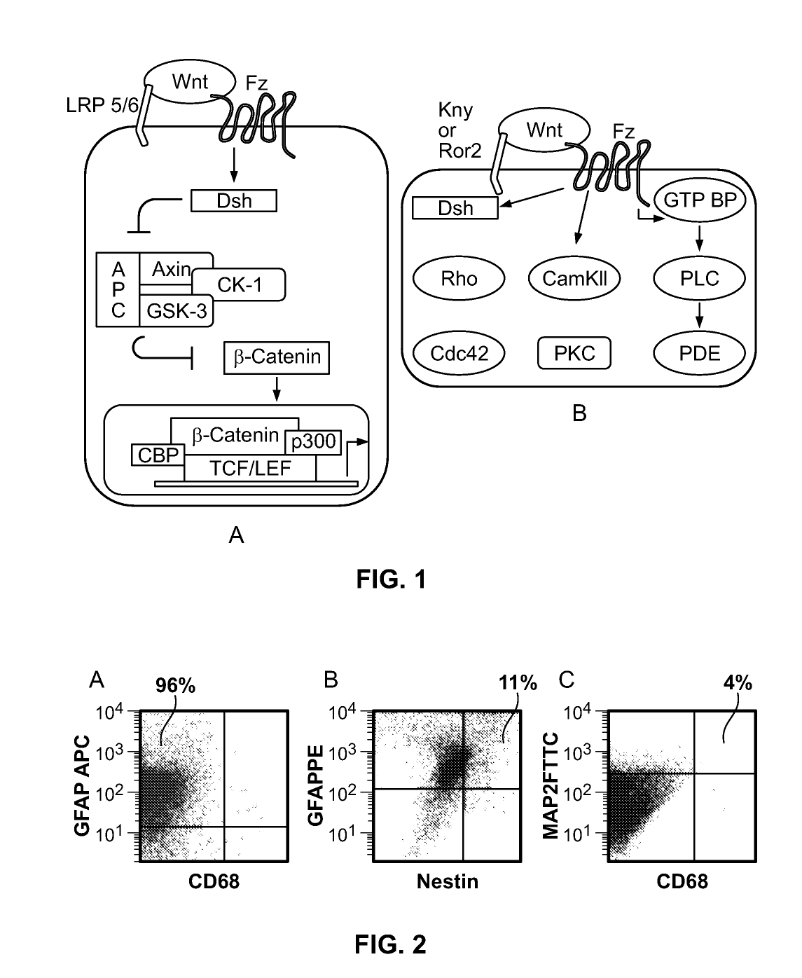

It was demonstrated herein that IFNγ markedly reduces basal Wnt signaling (FIG. 1). This is verified using SuperTOPflash, which is more sensitive than the commercially available TOPflash construct used to generate FIG. 11. Astrocytes were transfected with SuperTOP or SuperFOP along with pRL-TK (Renilla control plasmid), the cells are treated with IFNγ, and luciferase activity reflecting TCF-4 activation was assessed after controlling for Renilla activity. This experiment was conducted by priming the astrocytes with IFNγ first, which is the condition by which enhancement of HIV replication was observed, then transfecting the cells as indicated above and measuring luciferase activity after adjusting for Renilla activity. IFNγ inhibits basal levels of Wnt activity within astrocytes. Data indicate that IFNγ treatment leads to the inability of TCF-4 to immunoprecipitate with HIV TAR in infected cells.

### Example 15

### The Importance of Stat 1α in IFNγ-Mediated HIV Induction

This is determined by infecting cells that are knocked down for Stat but treated with IFNγ, if IFNγ requires Stat to induce HIV replication; it is expected that HIV replication is inhibited in those cultures. Any possible convergence between the IFNγ signaling pathway and the Wnt pathway is examined. This convergence is probable because i) IFNγ down-regulates c-myc, which is under the control of Wnt signaling and ii) IFNγ down-regulates Wnt signaling (FIG. 1). For these experiments, cells are transfected with SuperTOP or SuperFOP and Renilla with or without Stat 1alpha siRNA or irrelevant siRNA, which are commercially available (Upstate cells signal technology, Charlottesville, Va.). Control experiments is performed to ensure that Stat siRNA is effective using EMSA and Western blot. The transfected cells are either left untreated or treated with IFNγ. If Stat 1 alpha antagonizes β-catenin, knocking down Stat 1 alpha activity would reduce and/or abrogate the ability of IFNγ to inhibit Wnt activity, as assessed by this reporter assay readout. 2) Examining the direct impact of IFNγ on HIV LTR activity. Astrocytes are transfected with an LTR-Luciferase construct and treated pre- and post-transfection with IFNγ. A positive control is a co-transfection with Tat cDNA. TNFγ, a common LTR activator through NFκB is not appropriate because primary fetal astrocytes do not express its receptor (FIG. 3). Data indicates that IFNγ induces CCR1 and D6 expression, the latter was defined as a primary receptor for HIV on astrocytes, independent of CD4 co-expression. D6 is neutralized on astrocytes pre- and post-IFNγ treatment to assess if it is critical for HIV induction. The relief of the restriction is not likely to be at the level of HIV entry as untreated cells and IFNγ treated cells had similar level of early and late reverse transcripts (FIG. 6). 5) Evaluate the impact of IFNγ on endocytic trafficking. Although up-regulation of mannose receptor by IFNγ was not observed, it is still probable that IFNγ signaling may inhibit endocytic trafficking and thus removes this level of restriction to HIV, as reported. 6) Proteome technology may also be applied if the approaches above prove to be unfruitful. The protein profile in untreated and IFNγ treated cultures to define key proteins (known or novel) that may be linked to inhibition of HIV replication is assessed. These proteins are confirmed as inhibitors of HIV by inhibiting them with either cell permeable reagents or with siRNA technologies. Given that this proteome approach is cumbersome it is proposed as an alternative strategy, when all else is unsuccessful.

### Example 16

### Intrinsic Anti-HIV Molecular Mechanism and Permissive Targets

The intrinsic anti-HIV molecular mechanism disclosed herein can be extended to other permissive targets. Astrocytes provide a unique compartment with restricted HIV replication post-entry. This restriction, based on the present disclosure herein, appears to be at the level of Wnt signaling. It was shown that activation of Wnt signaling in primary CD3+ T cells by using LiCl led to a potent inhibition of HIV replication (FIG. 13). LiCl was recently described to have neuroprotective properties from HIV infected monocyte-derived macrophages in vitro and in the murine model of HIV encephalitis. Given that LiCl inhibits GSK-3alpha, leading to activation of β-catenin, demonstrates that Wnt activation is associated with HIV inhibition. LiCl-mediated inhibition of HIV replication in primary T cells provides a rationale to evaluate the potential of Wnt signaling in restricting HIV replication in cells other than astrocytes and especially in HIV permissive targets in the periphery. HIV suppression in the periphery eliminates and/or reduce NeuroAIDS complications. Wnt signaling pathway is a novel approach to regulate HIV replication. Although Wnt signaling is prominent in hematopoiesis and especially in thymopoiesis, its activity is diminished in mature lymphocytes. This observation coupled with the knowledge that activated lymphocytes support productive HIV replication is consistent with our hypothesis of an inverse relationship between Wnt signaling and productive HIV replication.

Experiments are conducted in parallel in primary human peripheral CD4+ T cells, monocyte derived-macrophages (MDMs), and a microglia cell line (HMG030, Clonexpress). MDMs serve as a model for microglia. HMG030, albeit a cell line, at this stage of the studies is informative to highlight differences in the Wnt pathway between a non-productive (astrocyte) and a productive (microglia) target of HIV infection. HMG030 is a human-derived microglia cell line that is phenoltypically (CD68+, CD 14+, esterase+, and HLA-DR+ after IFNγ treatment) and functionally (cytokine secretion in response to inflammatory signals) similar to primary microglial cells. Wnt activity is evaluated by measuring superTOP and SuperFOP luciferase activity with or without β-catenin siRNA.

Having any level of Wnt signaling in the cells inhibits HIV replication and without this signal HIV levels could be substantially higher. For this evaluation, MDMs and CD4+ T are isolated as described herein, used fresh, stimulated with LPS or anti-CD3/CD28, respectively, or left unstimulated. These cultures and HMG030 are infected with HIV. HIV replication is monitored by HIV reverse transcription initiation by PCR and p24 ELISA over time. Association between the level of Wnt signaling and HIV replication is assessed by Western blot for HIV p24 and β-catenin (active). At least three strains of HIV (T-tropic. M-Tropic, and a primary isolate) at high and low MOIs are tested in these experiments. The emergence of a negative association between β-catenin and HIV p24 level is expected, as evaluated by both p24 ELISA and Western blot but no impact on reverse transcription initiation, as the impact of the Wnt pathway in inhibiting HIV replication is believed to be at post-HIV entry and reverse transcription.

To assess the role of Wnt signaling as a novel anti-HIV therapeutic strategy, a comprehensive assessment of the role of Wnt activators and inhibitors in regulating HIV replication in MDMs, CD4+ T cells, and HMG030 is performed. A list of Wnt activators and inhibitors are disclosed herein. Activation of the Wnt pathway leads to inhibition of HIV in MDMs, CD4+ T cells, and HMG030. MDMs and CD4+ T cells are isolated as described herein, left inactivated or activated with LPS or anti-CD3/CD28, respectively, treated with various doses of the various Wnt activators. These activators are used at an effective dose that is not cytotoxic or cytostatic as evaluated by trypan blue exclusion assay, CFSE dye track loading, and PI staining. The cells are treated with the pre-determined dose of Wnt activators, infected with HIV, and the Wnt activator maintained throughout the duration of the experiment. HIV p24 is measured every 3 days for 14 days, as HIV infection in MDMs has slower kinetics than T cells. Various HIV strains (primary, T-topic, and M-tropic) and MOIs (high and low) are examined to establish any sensitivity to HIV strains or viral inoculums. The effectiveness of the various Wnt activators outlined in herein to restrict HIV replication are assessed in experiments that target identifying the effectiveness of the timing of exposure to the Wnt activator, whether added pre- or post-HIV infection or maintained in the culture throughout the duration of the experiment. A correlation is made between expression of HIV proteins within these infected cells and expression of active β-catenin by Western blotting, when necessary. Another approach for activating the Wnt pathway is via transfecting MDMs and CD4+ T cells with β-catenin-GFP cDNA with or without LPS or anti-CD3/CD28 co-stimulation, the cells infected with HIV, and HIV replication monitored by p24 intracellular immunostaining. Cells positive for GFP (expressing β-catenin cDNA) have low to undetectable intracellular p24 immunostaining as evaluated by FACS analysis. This indicates that Wnt signaling in these cells regulate HIV replication. For these experiments, specific Wnt pathway inhibitors as described herein are added to primary cells and HMG030 and the cells infected with HIV. HIV infection is monitored by ELISA and intracellular p24 expression. The HIV-GFP virus constructed lacking the identified TCF-4 binding sites in HIV are examined to directly associate its replicative capacity and ability of Wnt activators and inhibitors to impact its life cycle. Collectively, these studies are targeted to evaluate the association between Wnt signaling and HIV replication in primary targets for HIV replication and assess the potential of harnessing the Wnt pathway in pharmaceutical drug development for HIV.

### Example 17

### Restricting Multiple Variants of HIV

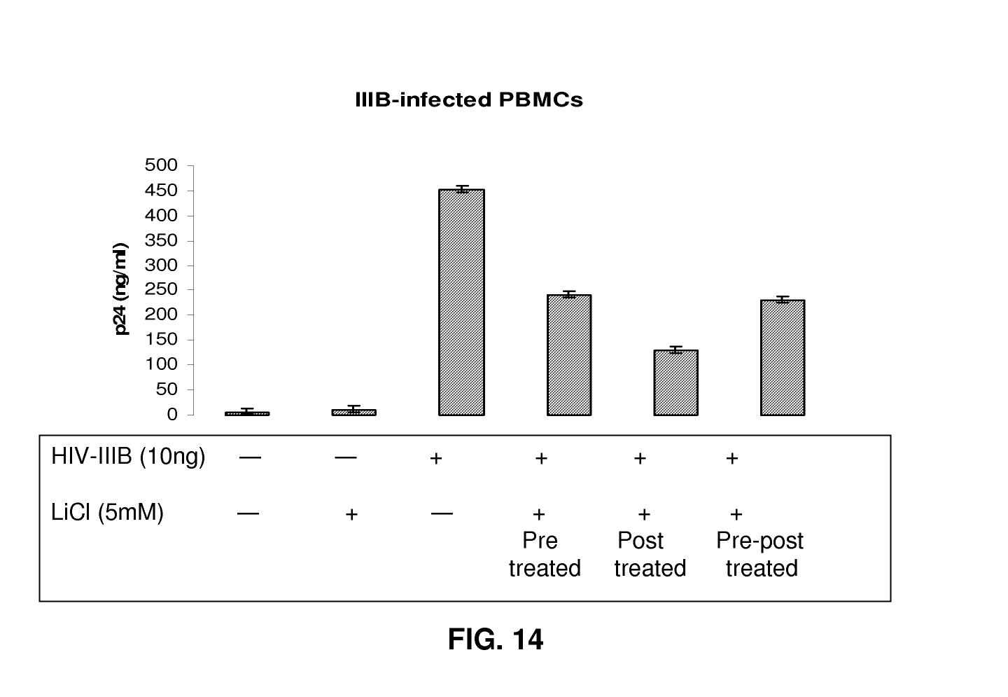

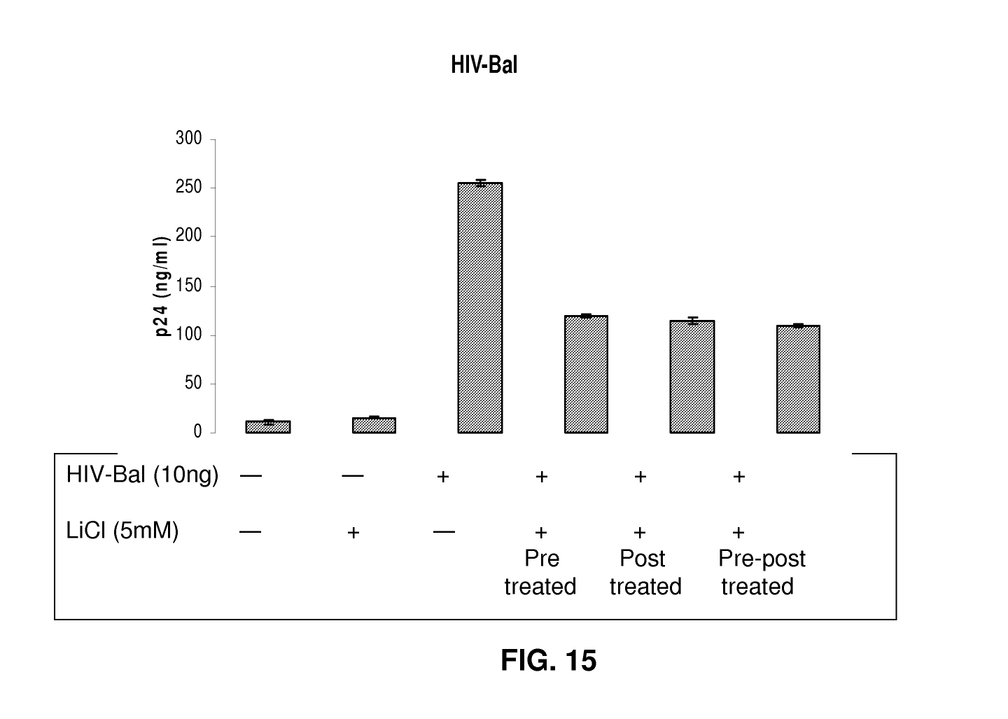

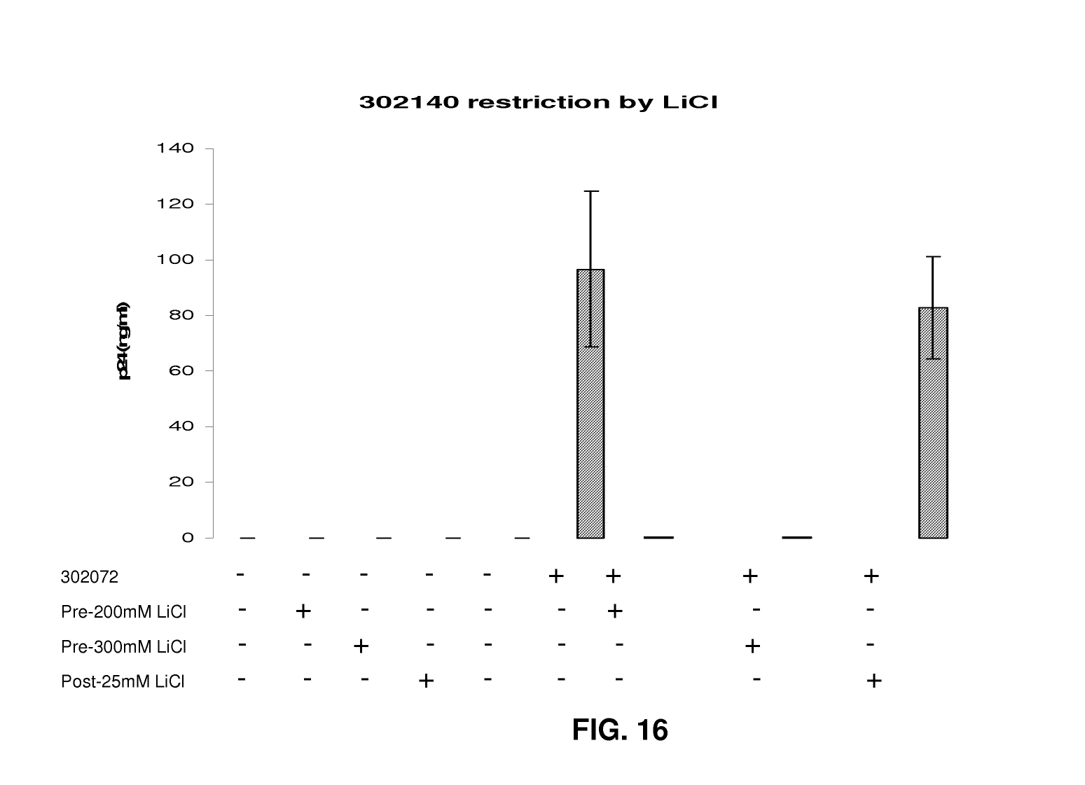

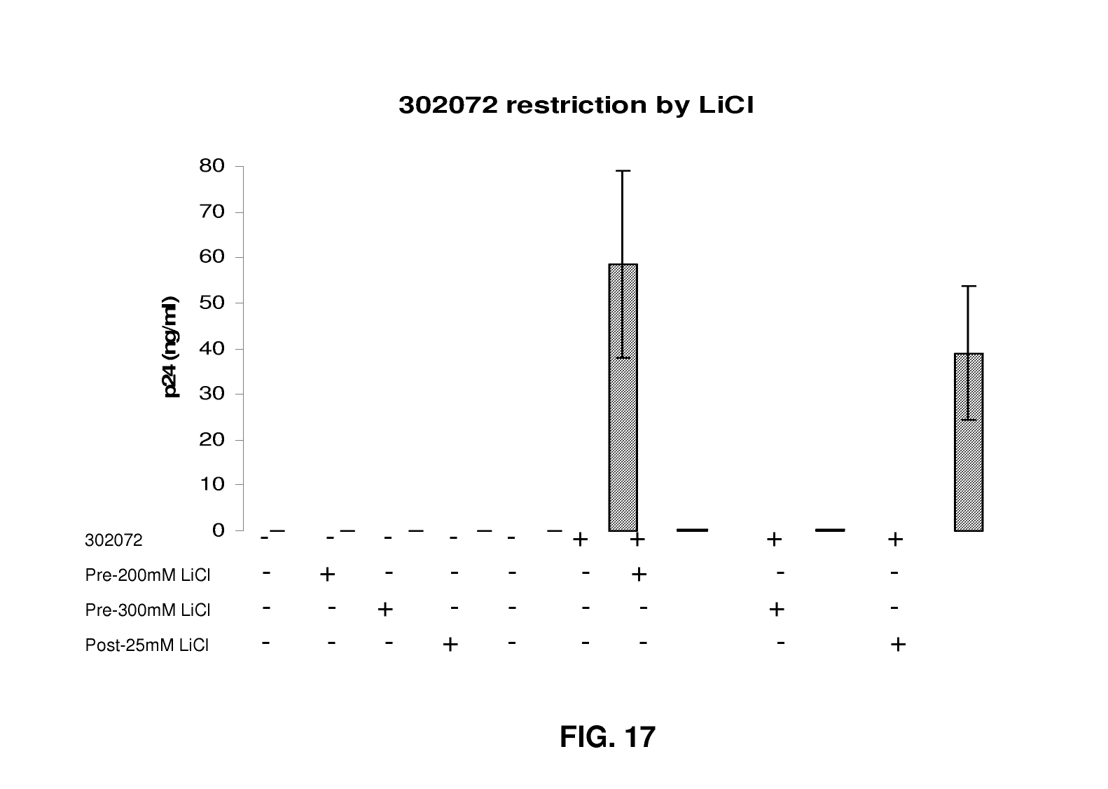

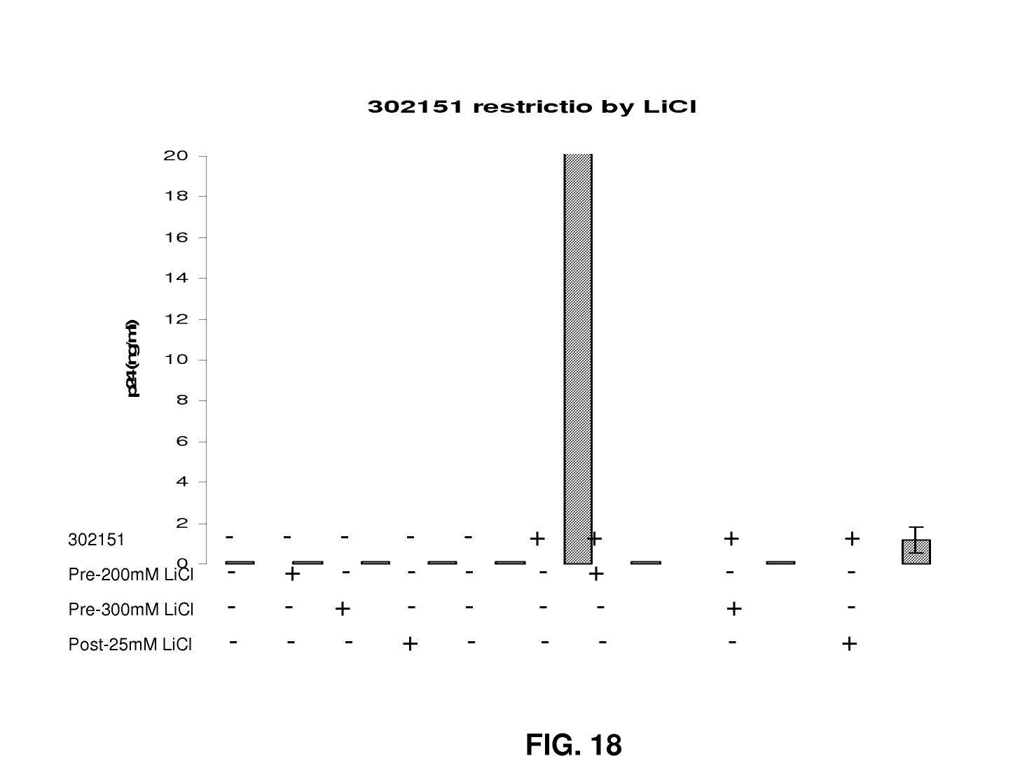

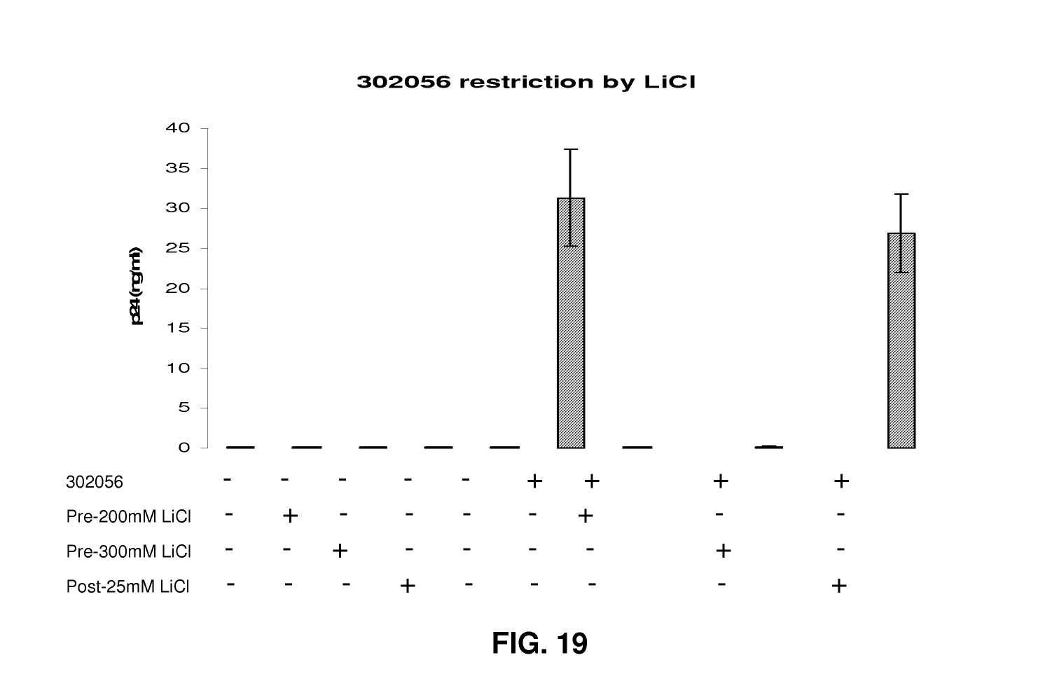

FIG. 14 shows that LiCl restricts replication of HIV-IIIB (X4). FIG. 15 shows that LiCl restricts replication of HIV-Bal (R5). FIGS. 16-19 demonstrate that LiCl inhibits a number of strains of HIV in PBMCs as indicated. PBMCs were treated with LiCl and infected with a number of primary HIV isolates. HIV replication was measured by p24 ELISA on day 7-post-infection.

### Example 18

### Wnt β-Catenin Represses HIV Replication

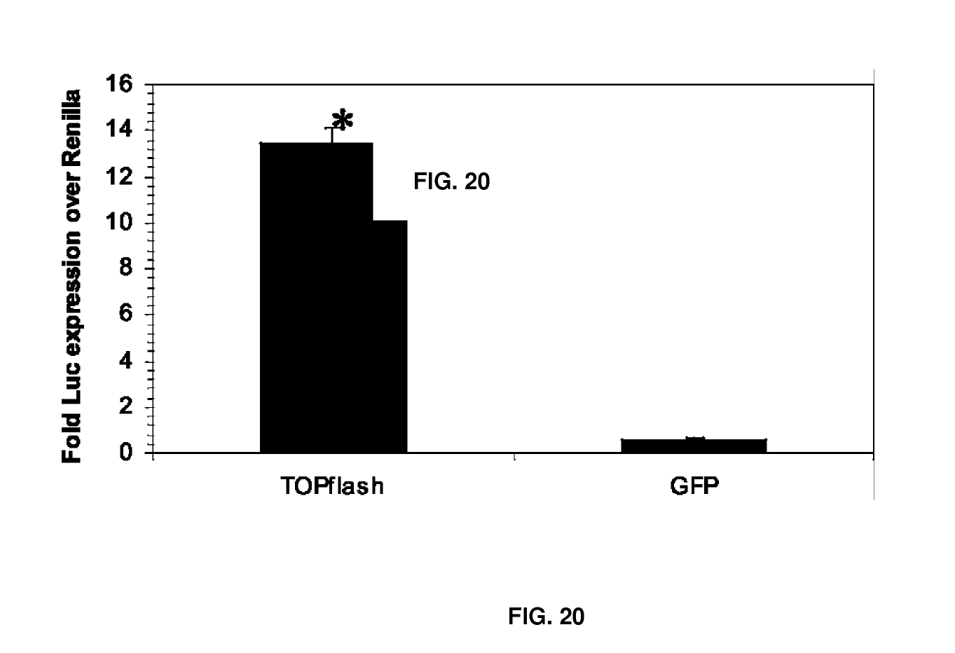

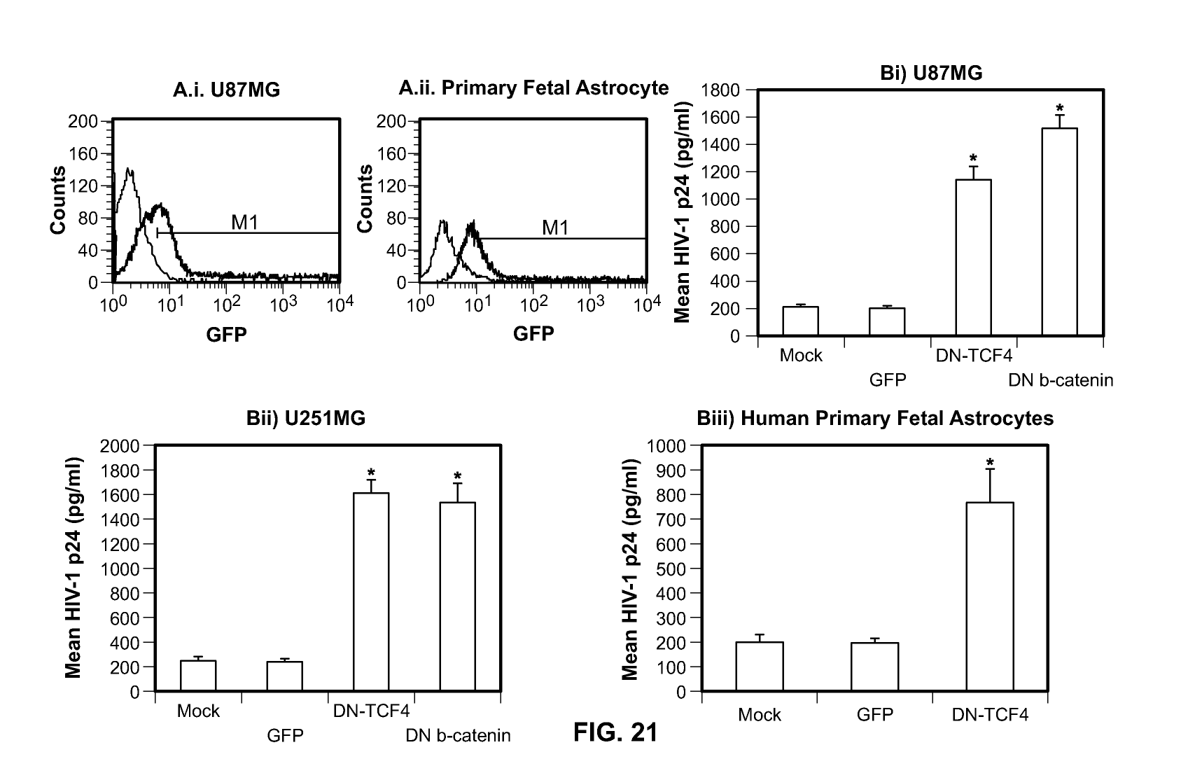

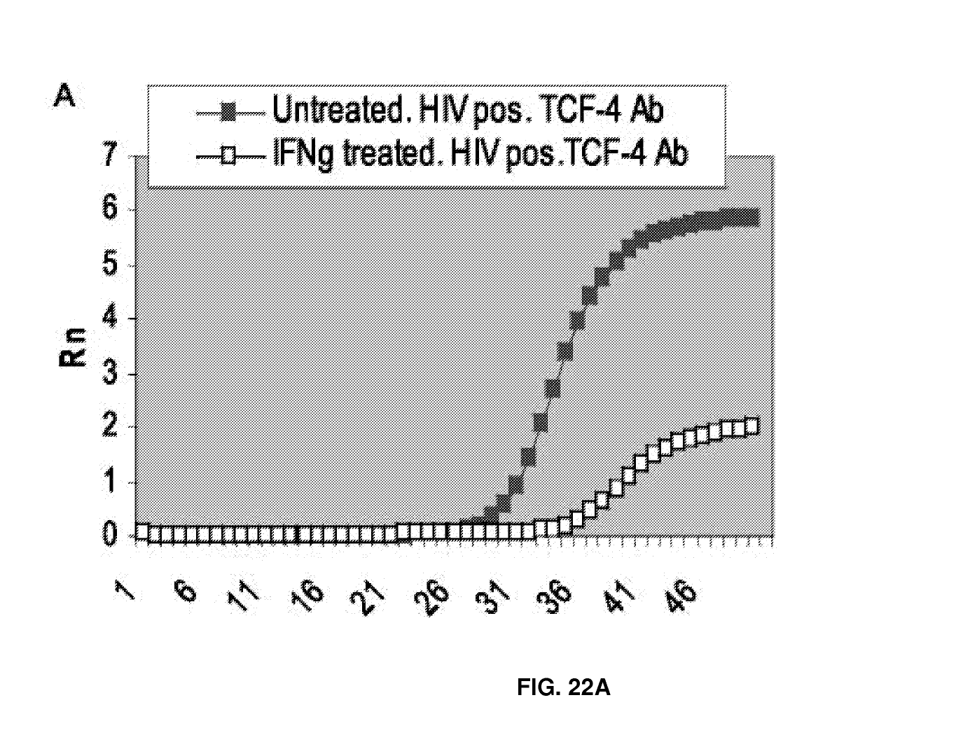

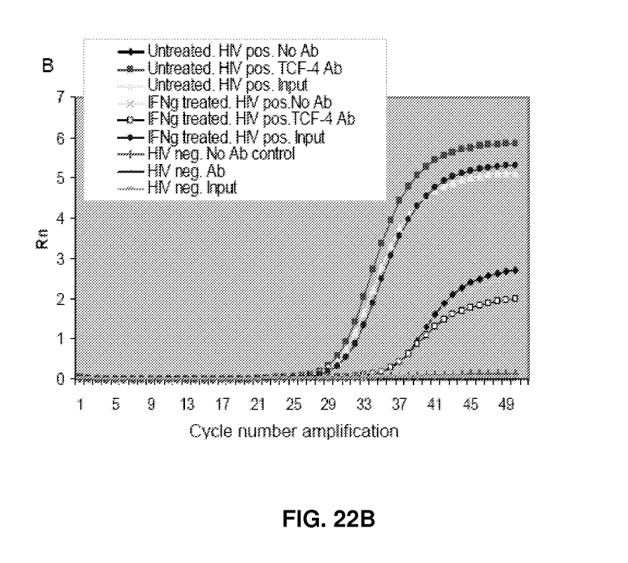

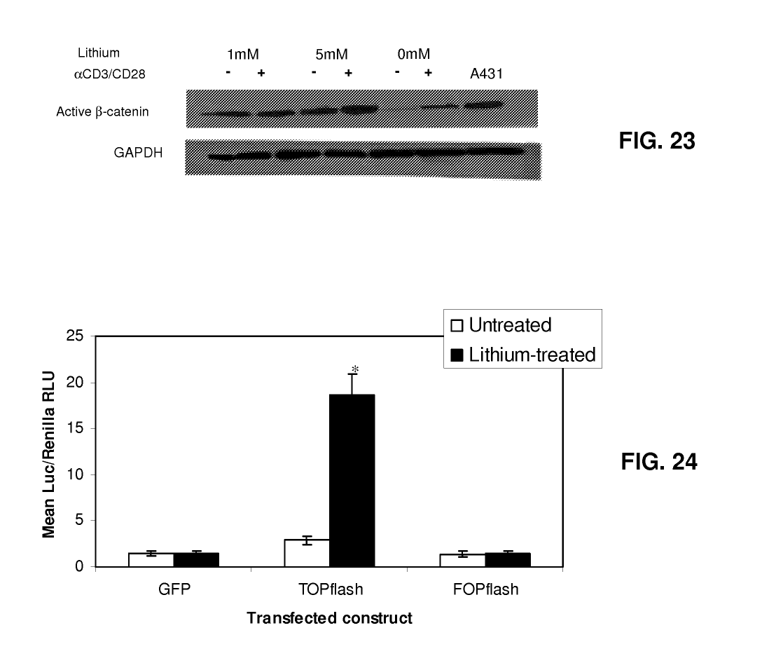

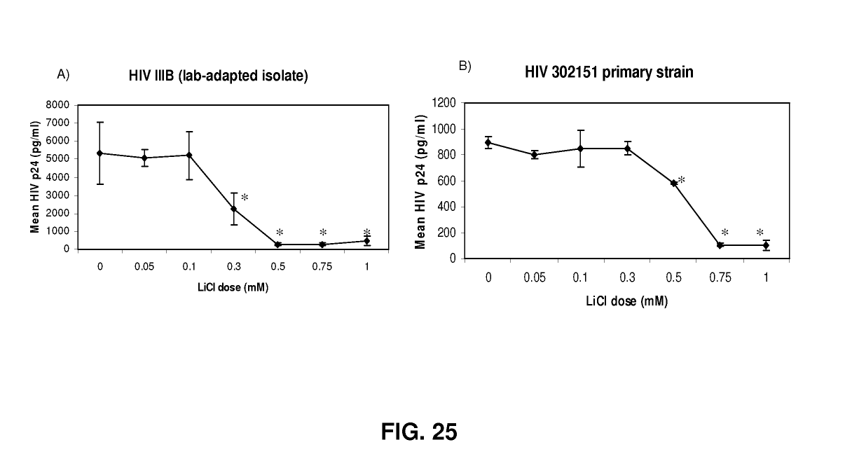

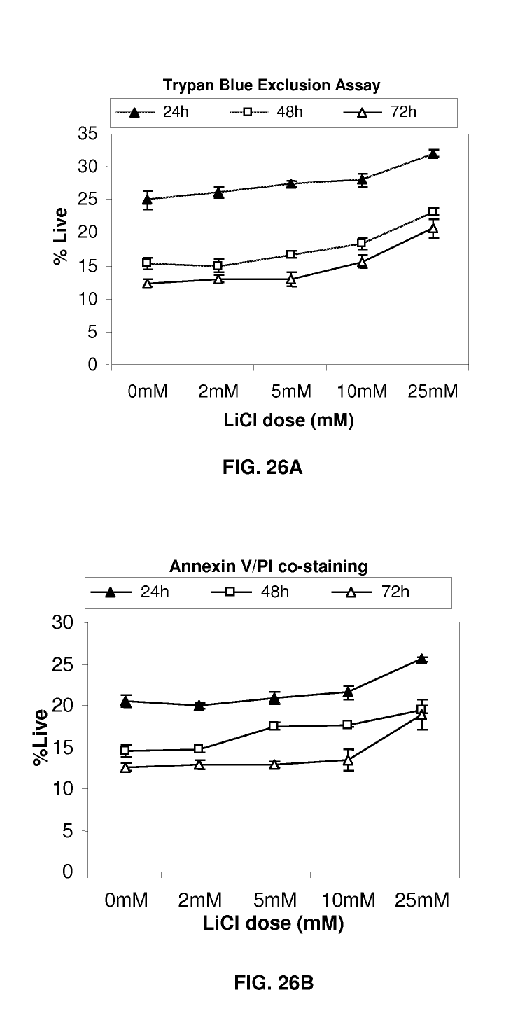

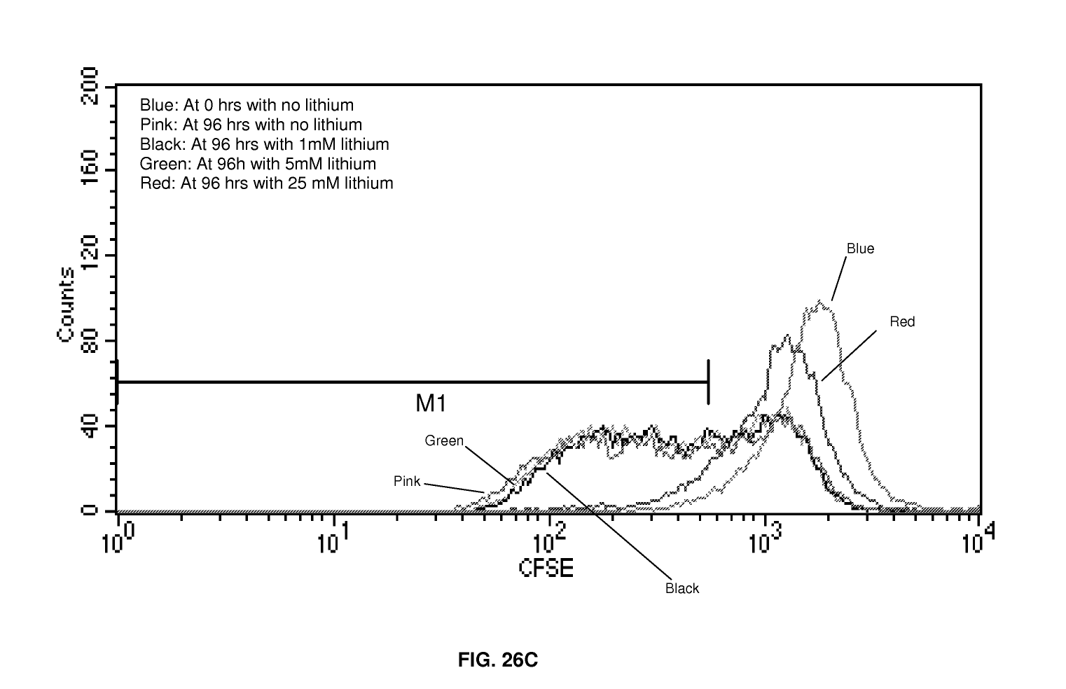

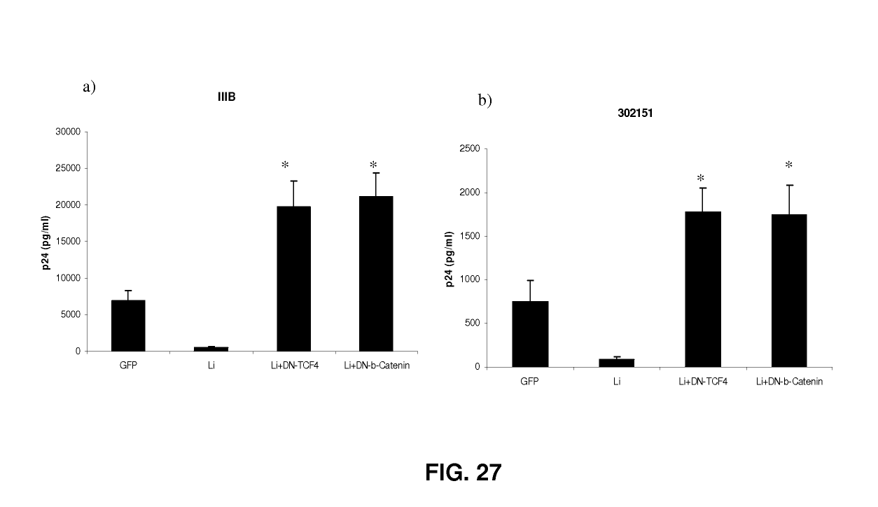

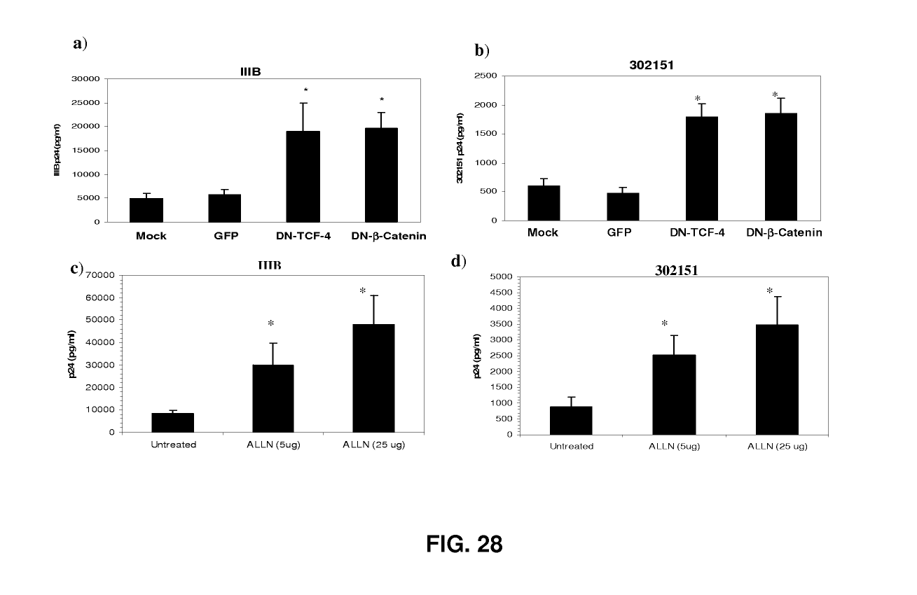

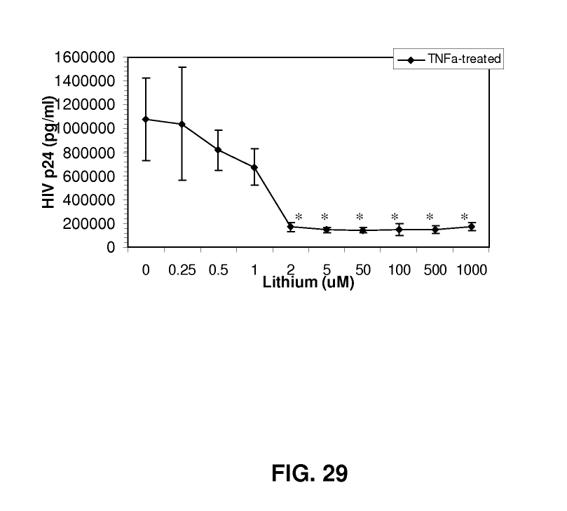

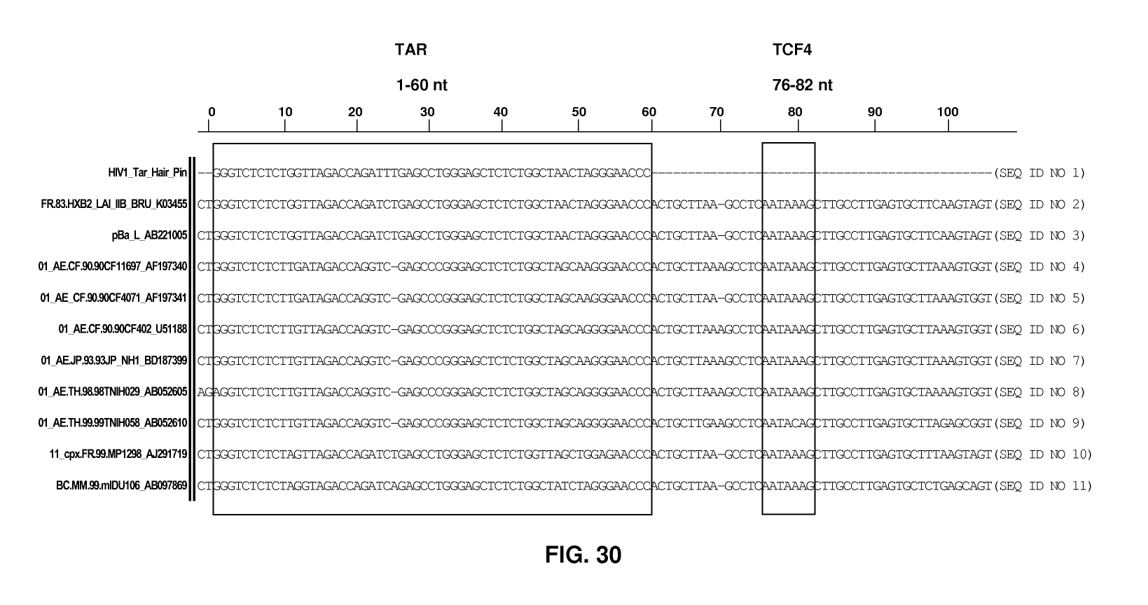

By modeling intracellular post-entry events that lead to HIV inhibition in astrocytes, the Wnt/β-catenin pathway was identified as a repressor of HIV replication. Utilizing a well known property of lithium to activate the Wnt/β-catenin pathway showed that lithium inhibits HIV in PBMCs. In astrocytes, using two astrocytoma cell lines and primary fetal astrocytes, restriction of HIV replication was mediated, in part, by endogenous Wnt signaling by demonstrating that: a) Wnt signaling is active in astrocytes (FIG. 20), b) Inhibition of the mediators of the Wnt/β-catenin pathway (TCF-4 or β-catenin) induce HIV replication (FIG. 21), and c) an association between TCF-4 and TARspanning region in infected cells that correlates with restricted replication of HIV (FIG. 22). In PBMCs, a) lithium induces Wnt signaling activity (FIGS. 23 and 24), b) the lithium dose to inhibit HIV (FIG. 25), c) is not cytotoxic nor cytostatic (FIG. 26) inhibiting basal Wnt activity induces HIV replication, demonstrating that the Wn/β-catenin pathway is a negative regulator of HIV replication (FIG. 27), and d) lithium inhibits re-activation of HIV in a latent cell line (J1.1) model system (FIG. 28).

Astrocytes, unlike resident microglia and infiltrating monocytes/macrophages, are resistant to productive/robust HIV replication. Resistance towards efficient HIV replication in astrocytes occurs at different steps of the HIV life cycle, including restrictions at HIV entry and post-entry events. Given that Wnt signaling is active in astrocytes (FIG. 20) and that the downstream effector of Wnt (TCF-4) is a repressor of LTR activation, the direct role of the canonical Wnt/β-catenin pathway on HIV replication in astrocytes was evaluated. U87MG, U251 MG, and primary human fetal astrocytes were transfected with either a dominant negative (DN) construct of TCF-4, a DN construct of E catenin, a construct of green fluorescence protein (GFP), or mock transfected. TCF-4 DN mutant is mutated in its β-catenin binding sites and is a repressor of TCF-4 activity. β-catenin DN construct lacks the N- and Cterminal domains for co-activation of transcription by β-catenin. Twenty four hours post-transfection, the cells were infected with HIVBal and seven days following infection HIV replication was measured by p24 ELISA. The efficiency of transfection at day three was at approximately 60% and 50% for U87MG/U25MG and HFA, respectively, as measured by GFP expression (FIG. 21 (A)). Inhibiting TCF-4 activity induced HIV replication in U87MG, U251, and primary fetal astrocytes by approximately 6-, 8-, and 4-fold, respectively, in comparison to cells transfected with the GFP plasmid alone (FIG. 21(B)). Inhibiting β-catenin also induced HIV replication in both cell lines to a similar level as that observed by TCF-4 inhibition. These data indicate that inhibition of TCF-4 activity removes some of the restriction to HIV replication in astrocytes and that the constrain on HIV replication is regulated partly by an active Wnt/β-catenin pathway.

TCF-4 is associated with TAR-spanning region in HIV infected astrocytes that are resistant to productive HIV replication: TCF-4, a Wnt signaling transcriptional factor, is defined as a transcriptional repressor of basal and Tat-mediated transactivation of the HIV LTR.

IFNγ priming of astrocytes leads to their permissiveness to productive HIV infection. To evaluate the role of TCF-4 in regulating HIV, the chromatin immunoprecipitation (ChIP) assay was used to determine if TCF-4 is associated with HIV TAR. Unlike electrophoresis mobility shift assay (EMSA), ChIP allows for the in vivo determination of the association between specific DNA-binding proteins (TCF-4 in this case) and specific region of the DNA (+1 to +153 in this case). To determine the association between TCF-4 and HIV, U87MG were left untreated or treated with IFNγ, infected with HIV, and cellular DNA was incubated with an antibody against TCF-4 to immunoprecipitate DNA that is associated with TCF-4. The immunoprecipitated DNA-protein complexes were dissociated and the DNA was amplified for HIV sequences. Detection of a PCR signal indicates association with TCF-4. Standard controls for the ChiP assay include an “Input control”, referring to amplification of the DNA before the immunoprecipitation step. Another control for ChIP is the “no antibody control”, referring to amplification of DNA after the immunoprecipitation step but without the addition of a specific antibody. Although HIV TAR is between +1 to +60, a larger fragment spanning R and U5 region (+1 to +153) was amplified since any protein binding in close proximity to TAR may interfere with TAR stereochemistry or DNA binding. This region (+1 to +153) is referred to as a TAR-spanning region. TCF-4 is immunoprecipitated with the TAR spanning region in untreated cultures but this association is absent when the cells are primed with IFNγ (FIG. 26). Taken together with the fact that untreated astrocytes do not support productive HIV replication, whereas IFNγ priming induces HIV replication suggests that there is an inverse relationship between TCF-4 association with TAR and HIV replication. These data point to a role of TCF-4 in regulating HIV replication in astrocytes.

Lithium induces β-catenin expression in PBMCs. The astrocyte model of intrinsic restriction to HIV replication indicated that active Wnt signaling contributes to HIV inhibition in these cells. To evaluate the potential of harnessing Wnt signaling to inhibit HIV in permissive targets, lithium was used to up-regulate the Wnt/β-catenin pathway and its impact on HIV replication in PBMCs was evaluated. PBMCs were isolated from healthy individuals, treated with LiCl (1 mM & 5 mM), and, because HIV replication is dependant on lymphocyte activation, they were stimulated with anti-CD3/CD28 at 1 Pg/ml each or left unstimulated as a control. Expression of active β-catenin (the dephosyrolated form) was evaluated by western blot at 24 hrs. LiCl induced active β-catenin in both unstimulated and stimulated PBMCs (FIG. 23). Anti-CD3/CD28 stimulation also induced β-catenin expression, but to a lower magnitude than in the presence of lithium.

To evaluate the extent to which lithium treatment containing four native TCF/LEF binding sites to measure Wnt activity), FOPflash (a construct containing four mutated induced Wnt signaling, PBMCs were transfected with Active β-catenin the TOPflash (a construct LEF/TCF binding sites), GFP, or Renilla. PBMCs were left untreated or treated with LiCl at 1 mM. Luciferase activity was evaluated 24 hrs post-transfection and normalized to Renilla. Lithium treatment up-regulated Wnt signaling by four-fold in TOPflash-transfected but not in FOPflash-transfected cultures (FIG. 24). These data confirm the observation that lithium induces Wnt/β-catenin signaling and demonstrates that it can also occur in PBMCs.

Lithium inhibits HIV replication in PBMCs: Lithium induces the Wnt pathway by inhibiting GSK3 E, leading to a dephosphorylated (active) β-catenin. This lithium-mediated induction of Wnt/β-catenin in PBMCs (FIGS. 23 and 24). To assess if activation of the Wnt pathway can restrict HIV replication in permissive targets, the ability of lithium to inhibit HIV replication was evaluated in PBMCs. PBMCs were stimulated with D-CD3/CD28 and treated with LiCl at various doses (0-1 mM) then infected with either a laboratory adapted strain (HIV IIIB) or a primary isolate of HIV (302151). HIV replication was evaluated by measuring HIV p24 core protein at day seven post-infection using conventional ELISA. LiCl inhibited HIV IIIB by 50% at 0.3 mM and >90% inhibition by 0.5 mM (FIG. 25A). Inhibition of the primary isolate by LiCl was also observed but it required a higher dose of LiCl at 0.75 mM to reach >90% inhibition (FIG. 25B). These data indicate that treating PBMCs with LiCl leads to substantial inhibition of HIV replication of both primary and laboratory isolates of HIV.

Given that lithium inhibited HIV replication in PBMCs, the impact of lithium was evaluated on cell viability, to exclude indirect inhibition by impacting cell survival. PBMCs were stimulated with anti-CD3/CD28 and treated with LiCl at 0-25 mM. Cell viability was evaluated at 24, 48, and 72 hrs by the trypan blue exclusion assay and co-staining for annexin V and propidium iodide (PI). At all of the doses tested, LiCl did not impact cell viability as determined by the trypan blue exclusion assay and staining for necrotic (PI+) and apoptotic (annexin V+) cells (FIG. 26), even at doses that far exceeded the demonstrated dose to inhibit HIV.

To evaluate the impact of lithium at the dose shown to inhibit HIV replication (1 mM) on cell turnover, PBMCs were stimulated with anti-CD3/CD28 for 48 hrs then loaded with carboxyfluorescein succinimidyl ester (CFSE) and treated with 1, 5, and 25 mM LiCl or left without lithium treatment. Dilution of CFSE was measured by flow cytometry at 96 hrs. At a lower dose of LiCl (1 mM & 5 mM) it had no effect on cell turnover but at a higher do se (25 mM) LiCl reduced cell division (FIG. 27). Similar data trends were observed at 48 and 72 hrs.

Inhibiting basal Wnt activity induces greater level of HIV replication in PBM CsPBMC activation by anti-CD3/CD28 treatment induces β-catenin expression, as demonstrated in (FIG. 20). The impact of inhibiting basal Wnt/β-catenin signaling in PBMCs on HIV replication (IIIB and primary strain 302151) was evaluated. Three complementary approaches were utilized to inhibit basal/en dogenous Wnt signaling in PBMCs: 1) transfecting PBMCs with a dominant negative (DN) mutant of TCF-4, 2) transfecting PBMCs with a DN mutant of E-catenin, 3) or treating PBMCs with a cell permeable N-acetyl-Leu-Leu-norleucinal (ALLN) peptide which inhibits proteasome-mediated proteolysis and induces the accumulation of phosphorylated, ubiquitinated β-catenin. Inhibiting the activity of endogenous TCF-4 or β-catenin induced HIV replication by approximately four-fold in comparison to mock or GFP-transfected cultures, independent of the HIV isolate used (FIG. 28). Further, treating PBMCs with ALLN peptide at either 5 or 25 M also induced HIV IIIB and 30215 replication by three- and five-fold, respectively. Wnt/β-catenin signaling is a repressor of HIV replication.

Extensive data demonstrated that Wnt/β-catenin signaling is associated with restricted HIV replication in astrocytes and even affects the extent to which HIV replicates in lymphocytes. Activation of this pathway by lithium is a viable approach to restrict HIV replication in multiple cell targets.

### Example 19

### Lithium Inhibits HIV in Peripheral Blood Mononuclear Cells by Activating the Wnt/β-Catenin Pathway

To evaluate the potential of harnessing Wnt/β-catenin signaling to inhibit HIV in permissive targets, lithium was used to up-regulate the Wnt/β-catenin pathway. The impact on HIV replication in PBMCs was evaluated. Lithium is an inducer of the Wnt/β-catenin pathway, as reported in oocytes from invertebrate animals and in human thyrocytes. Lithium induces Wnt/β-catenin signaling in PBMCs by measuring protein expression of the central mediator of this pathway (β-catenin) and the transcriptional activity of the down stream effector of the Wnt/β-catenin signaling pathway (TCF/LEF transcriptional factors). To evaluate the impact of lithium on β-catenin expression, PBMCs were isolated from healthy individuals and treated with LiCl at its therapeutic recommended plasma level (1 mM) and at a higher dose (5 mM). Because HIV replication is dependant on lymphocyte activation, they were stimulated with anti-CD3/CD28 at 1 mg/ml each or left unstimulated as a control. Expression of active β-catenin (the dephosphorylated form) was evaluated by western blot at 24 hrs. A431 cells constitutively express β-catenin and their lysate was used as a positive control. The housekeeping gene glyceraldehyde-3-phosphate dehydrogenase (GAPDH) was used as a loading control. LiCl induced active β-catenin in both unstimulated and stimulated PBMCs (FIG. 23). Interestingly, anti-CD3/CD28 stimulation also induced β-catenin expression, but to a lower magnitude than in the presence of lithium. These data demonstrate that basal level of β-catenin in PBMCs is low in resting cells but that it can be up-regulated in response to T cell activation (anti-CD3/CD28 co-stimulation) and more potently in response to lithium treatment. These findings not only underscore the ability of lithium to induce β-catenin in PBMCs but also suggest that β-catenin may play a role in the function of T cells post-development and in response to T cell activation.

To measure the transcriptional activity of the down stream effector of the Wnt/β-catenin signaling pathway, TCF/LEF transcriptional factors, PBMCs from healthy donors were transfected with TOPflash, a luciferase construct containing four native TCF/LEF binding sites (TOPflash) or its negative control counterpart (FOPflash) containing four mutated LEF/TCF binding sites. A construct of green fluorescent protein (GFP) was used as the negative control. PBMCs were also co-transfected with an internal plasmid control (Renilla). Transfected PBMCs were left untreated or treated with LiCl at 1 mM and 5 mM. Luciferase activity was evaluated 24 hrs post-transfection and normalized to Renilla. Basal expression of TCF/LEF was low in PBMCs, as measured by TCF/LEF transcriptional activity that was similar to the GFP control (FIG. 24) Lithium treatment up-regulated Wnt signaling by 4-fold in TOPflash-transfected but not in FOPflash-transfected cultures (FIG. 24). These data demonstrate that lithium induces Wnt/β-catenin signaling in PBMCs.

Lithium activates Wnt/β-catenin in PBMCs. Lithium can modulate HIV replication in PBMCs. PBMCs were stimulated with α-CD3/CD28 and treated with LiCl at various doses (0-1 mM) then infected with either a CXCR-4 lab-adapted isolate of HIV (HIV IIIB)(18) or a CCR-5 utilizing primary isolate of HIV 302151 (Also known as HIV 93US151) (NIH AIDS research and reference reagent program, Germantown, Md.). HIV replication was evaluated by measuring HIV p24 core protein at day seven post-infection using conventional ELISA. LiCl inhibited HIV IIIB by 50% at 0.3 mM and >90% inhibition by 0.5 mM (FIG. 25). Inhibition of the primary isolate by LiCl was also observed but it required a higher dose of LiCl at 0.75 mM to reach >90% inhibition (FIG. 25). These data indicate that treating PBMCs with LiCl leads to substantial inhibition of HIV replication of both primary and laboratory isolates of HIV.

To exclude that the ability of lithium to inhibit HIV may be mediated by cytotoxic or cytostatic effects, the impact of lithium was evaluated on cell viability and proliferation. PBMCs were stimulated with a-CD3/CD28 and treated with LiCl at 0-25 mM. Cell viability was evaluated at 24, 48, and 72 hrs by the trypan blue exclusion assay and co-staining for annexin V and propidium iodide (PI). At all of the doses tested, LiCl did not impact cell viability as determined by the trypan blue exclusion assay and staining for necrotic (PI+) and apoptotic (annexin V+) cells (FIGS. 26 A & B), even at doses that far exceeded the >0.1 mM dose demonstrated to inhibit HIV. To evaluate the impact of lithium on cell proliferation, PBMCs were stimulated with a-CD3/CD28 for 48 hrs then loaded with carboxyfluorescein succinimidyl ester (CFSE) and treated with 1, 5, and 25 mM LiCl or left without lithium treatment. Dilution of CFSE was measured by flow cytometry at 96 hrs. At lower doses (1 mM & 5 mM), lithium had no effect on cell turnover but at a higher dose (25 mM) lithium reduced cell division (FIG. 26 C). Inhibition of cell turnover at a higher lithium dose is consistent with a number of reports, albeit not in PBMCs, demonstrating that at doses exceeding 10 mM, lithium causes cell cycle arrest. At least in one observation based on human hepatic cells, the lithium inhibitory effect on cell proliferation was independent of its effects on the Wnt/β-catenin pathway but rather down regulation of PKB/Akt and cyclin E proteins. These data demonstrate that lithium inhibits >90% of HIV at 0.5-0.75 mM, depending on the HIV isolate used, which is a dose that is within the recommended therapeutic plasma level of lithium (0.6-1.2 mM) and is not cytotoxic nor cytostatic.

At therapeutic plasma levels, lithium inhibits the activity of both the α and β isoforms of GSK3, resulting in the activation of the Wnt signaling pathway. However, at higher concentrations (4-5 mM) lithium inhibits inositol monophosphatase (IMPase) and inositol polyphosphatase, leading to a decreased IP₃ response. Because the dose of lithium determined to inhibit HIV was ideal for Wnt/β-catenin activation and not IP3 effects and because Wnt/β-catenin represses HIV replication post entry in a non-HIV permissive cell target, it was determined whether lithium inhibition of HIV is mediated through its activation of the Wnt/β-catenin pathway. Towards this goal, PBMCs from healthy donors were stimulated with α-CD3/CD28 for 48 h then transfected with a dominant negative (DN) construct of T cell factor 4 (TCF-4), β-catenin, or green fluorescent protein (GFP) then infected with HIV IIIB or primary strain 302151 (93US151). After 24 hrs, lithium at 1 mM was added to the cultures and HIV p24 ELISA was performed on day 7 post-infection. The TCF-4 dominant negative mutant inhibits the interaction between TCF-4 and β-catenin. It lacks the N-terminal sequences required for β-catenin binding but retains DNA binding activity and functions in a dominant negative manner. TCF-4 activity was inhibited because this transcriptional factor is a repressor of HIV replication. Inhibiting either the down-stream effector of the Wnt signaling pathway (TCF-4) or the central mediator of this pathway (β-catenin) using their respective dominant negative mutant constructs abrogated the ability of lithium to inhibit the replication of HIV strain IIIB or the primary HIV strain 302151. Lithium inhibits HIV through the Wnt/β-catenin pathway.

The level of HIV replication is enhanced with inhibition of the Wnt/β-catenin pathway, by approximately 4-fold, suggesting that inhibiting the endogenous/basal level of Wnt/β-catenin in PBMCs enhances HIV replication. The role of endogenous Wnt/b-catenin signaling in regulation of HIV replication was explored. Specifically, stimulated PBMCs were transfected with either DN-TCF-4, DN-β-catenin, GFP, or treated with a cell permeable N-acetyl-Leu-Leu-norleucinal (ALLN) peptide which inhibits proteasome-mediated proteolysis and induces the accumulation of phosphorylated, ubiquitinated β-catenin. Twenty-four hours post-transfection or ALLN treatment, the cells were infected with HIV IIIB or strain 302151 and HIV replication was measured by p24 ELISA on day 7. Efficiency of transfection at day 2 post-transfection was approximately 59% as measured by GFP expression. Inhibiting the activity of endogenous TCF-4 or catenin induced HIV replication by approximately four-fold in comparison to mock or GFP-transfected cultures, independent of the HIV isolate used. Further, treating HIV-infected PBMCs with ALLN peptide at either 5 or 25 mM also induced HIV IIIB and 30215 replication by three- and five-fold, respectively, in comparison to none-ALLN treated cultures. These data demonstrate that endogenous Wnt/β-catenin activity is an intrinsic host mechanism that represses HIV replication, removing this repression enhances HIV replication in PBMCs and up-regulating this pathway by lithium treatment reduces HIV replication.

Transactivation of the HIV LTR in a β-catenin-independent manner. While our data is consistent with the observation that TCF-4 is a repressor of HIV replication, in PBMCs, we provided evidence to indicate that this repression is β-catenin-dependant, as demonstrated by abrogation of the inhibitory effect when β-catenin activity was inhibited though its respective dominant negative mutant (FIGS. 27 & 28). These data indicate that integral components of Wnt pathway, including β-catenin, are critical in repressing HIV replication in PBMCs.

To evaluate whether lithium can also inhibit re-activation of latent HIV, we used the J1.1 latent infection cell model. J1.1 is a latently infected Jurkat cell line whereby HIV is reactivated with TNFa or mitogen stimulation (25). J1.1 cells were treated with TNFa (100 U/ml) with or without lithium (0.25-10000M) and HIV replication evaluated by p24 ELISA three days post-stimulation. Lithium inhibited TNFa-mediated induction of HIV replication by four logs starting at 2 mM. The data presented showing that lithium inhibited TNFa-mediated induction of HIV in J1.1 cell (an NF-kB-dependant mechanism, at a much lower dose (2 mM) than de novo infection (1 mM) suggest that lithium may also be inhibiting NF-kB. GSK-3b induces NF-kB function. Therefore since lithium inhibits GSK-3b, this may lead to NF-kB inhibition as well. In J1.1, lithium may be activating a repressor (Wnt/β-catenin) and inhibiting an activator (NF-kB) of HIV, leading to potent suppression at a lower dose.

Lithium prevents gp120- and Tat induced HIV neurodegeneration in vitro and increases soluble TNFa receptor, which absorbs the neurotoxic TNFa cytokine. In a small pilot study evaluating the therapeutic benefit of lithium in improving cognition in HIV+ patients, within 12 weeks of low-oral dose lithium therapy, the cognition score improved in all participants (n=8) and became unimpaired in 75% of the enrolled patients. Further, lithium (600-1200 mg/day) was well tolerated in this small clinical study with no grade 3 or 4 adverse events or withdraw from study because of adverse effects.

### Example 21

### HIV-Restricted Replication in Astrocytes, IFNγ Regulation

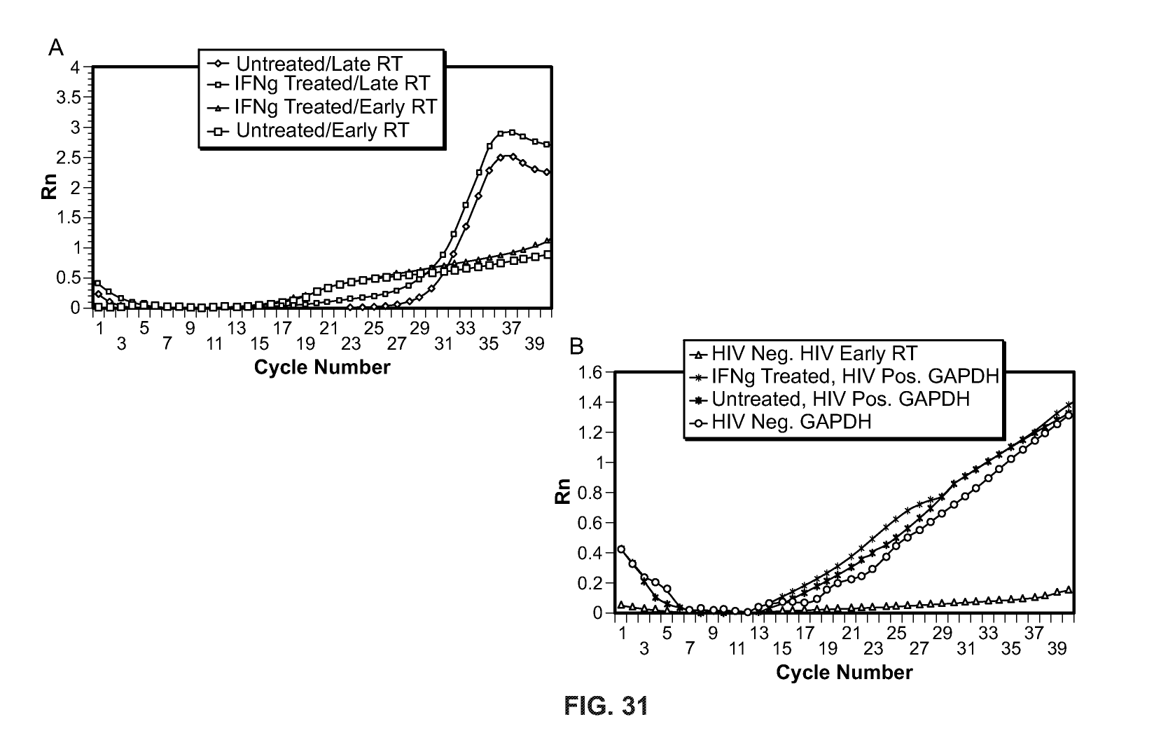

Levels of HIV reverse transcription are similar between untreated and IFN-γ-treated astrocytes. IFN-γ pretreatment of astrocytes leads to induction of HIV replication. To evaluate the mechanism of HIV restriction in astrocytes and the mechanism by which IFN-γ regulates this restriction, the rates of HIV early and late reverse transcription were compared between untreated and IFN-γ-treated astrocytes. U87MG cells were left untreated or pretreated with IFN-γ for 24 h and then infected with HIVBAL. Unbound virus was removed by trypsinization. Early HIV reverse transcription was evaluated by real-time PCR at 24 h postinfection by amplification with the R/U5 primer pair, which detects negative-strand “strong-stop” DNA indicative of reverse transcription initiation). Late HIV reverse transcription was measured 96 h postinfection using primer pair R/5NC, which amplifies late reverse transcripts containing positive-strand DNA after the second template switch beyond the primer binding site. The glyceraldehyde-3-phosphate dehydrogenase (GAPDH) housekeeping gene was coamplified as an internal control. The levels of early and late reverse transcription DNA amplification were similar between cultures left untreated and those treated with IFN-γ (FIG. 31). These data indicate that HIV enters astrocytes and undergoes early and late reverse transcription but that enhanced virion entry or an accelerated rate of reverse transcription post-IFN-γ treatment is not a likely contributing factor for IFN-γ-mediated induction of HIV replication.

The HIV LTR in untreated astrocytes is associated with acetylated histones indicative of regions of active gene transcription. Given that HIV undergoes early and late reverse transcription in astrocytes and yet is well documented to be restricted in productive HIV replication, the status of histone modification of the HIV LTR was evaluated in untreated astrocytes. Several histones wrap around the DNA, and when deacetylated they lead to chromatin condensation and are associated with inactive genes. However, once the histones are acetylated, the chromatin structure is modified, becoming accessible to replication enzymes. Acetylated histones correlate with regions of active gene expression. For instance, multiple acetylated lysine residues have been identified in the N-terminal domain of H2B that correlate with gene-specific transcriptional activation. These modifications aid in the structural and functional properties of nucleosome and nucleosomal arrays seen in various activated cellular genes. Inhibition of HIV replication in astrocytes may be due to its association with histone modification (i.e., deacetylation), leading to inactive gene transcription. To assess this, a ChIP assay was performed on U87MG cells infected with HIV and DNA was analyzed 96 h postinfection. An antibody to acetylated histone H2B (Lys 5/12/15/20) was used to immunoprecipitate cellular DNA from HIV-infected astrocytes. These complexes were then dissociated, and the DNA was amplified for the HIV LTR at the region between −460 and −206 and the R and U5 regions including TAR between +1 and +153 from the transcription initiation site. Detection of a signal indicates association with the acetylated protein. The two regions amplified for LTR and the TAR-spanning region were associated with acetylated histones (FIG. 8), as their respective DNA was amplified post-IP with the acetylated-histone-recognizing antibody. These data indicate that the HIV promoters at two loci (+1 to +153 and +460 to +206) are associated with transcription-ready complexes. The fact that the immunoprecipitated DNA contained both HIV-specific genes and the housekeeping gene (GAPDH), suggests that the HIV DNA amplified in these cultures represented integrated DNA. These data further suggest that the block to restricted HIV replication in astrocytes is not caused by condensed chromatin association and subsequent inhibition of active HIV gene transcription.

An inverse association between TCF-4 IP with HIV TAR containing region and productive HIV replication in astrocytes. TCF-4, a Wnt signaling transcriptional factor, is defined as a transcriptional repressor of the HIV LTR. Although the exact mechanism of TCF-4-mediated inhibition of the HIV LTR is not clearly delineated, its association with HIV Tat may inhibit Tat transactivation of the HIV LTR. To evaluate the effect of TCF-4 on both HIV restriction in astrocytes and IFN-γ-mediated induction of HIV replication in astrocytes, the association between TCF-4 and the HIV TAR-containing region was examined with or without IFN-γ treatment. ChIP was performed on U87MG cells left untreated or treated with IFN-γ, infected with HIV, chromatin immunoprecipitated with a TCF-4-specific antibody, and DNA amplified for HIV between +1 and +153 bp. TCF-4 is immunoprecipitated with TAR in untreated cultures but that this association is absent when the cells are primed with IFN-γ (FIG. 9). Untreated astrocytes do not support productive HIV replication, whereas IFN-γ priming induces HIV replication, suggesting that there is an inverse relationship between TCF-4 association with TAR-containing region and HIV replication. There is likely a role for TCF-4 in regulating HIV replication in astrocytes.

TCF-4 inhibition reverses the restriction of HIV replication in untreated/non-cytokine-primed U87MG cells and primary HFA. Given the observed inverse relationship between HIV replication in astrocytes and TCF-4 and TAR association, whether via direct or indirect binding, the direct role of TCF-4 in HIV replication was evaluated by using a dominant-negative mutant of TCF-4. This dominant-negative TCF-4 mutant is mutated in its β-catenin binding site and is a repressor of TCF-4 activity through the canonical α-catenin-dependent pathway. U87MG cells and primary HFA were transfected with a TCF-4 dominant-negative mutant or GFP-encoding plasmid. The total amount of DNA remained constant between the cultures. Twenty-four hours posttransfection, U87MG cells and HFA were infected with HIV, and HIV replication was measured by p24 ELISA on day 7. The efficiency of transfection at day 3 posttransfection was approximately 50% and 35% for U87MG cells and HFA, respectively, as measured by GFP expression (FIG. 10). Inhibiting TCF-4 in U87MG cells and HFA modulated HIV replication by three-fold in comparison to cells transfected with the GFP-encoding plasmid alone (FIG. 10B). This level of HIV replication after transfection of astrocytes with dominant-negative TCF-4 is similar to that achieved by priming the cells with IFN-γ. Priming the cells with IFN-γ prior to transfection with dominant-negative TCF-4 did not result in a higher rate of HIV replication than that in cultures treated with IFN-γ alone. These data indicate that inhibition of TCF-4 activity removes the restriction of HIV replication in astrocytes.

Active Wnt signaling in astrocytes and its inhibition by IFN-γ. Although inhibition of TCF-4 by the dominant-negative mutant stresses the importance of active TCF-4 in restricting HIV replication, it was not informative regarding the mechanism by which IFN-γ overcomes astrocyte restriction to productive HIV replication. To evaluate the impact of IFN-γ on TCF-4 activity in regulating HIV replication, U87MG cells were left untreated or IFN-γ treated and then transfected with either a TCF-4 luciferase construct (TOPflash) or a GFP construct and cultured with or without IFN-γ. The TCF-4 reporter construct is an indicator of basal and inducible levels of Wnt signaling. Basal TCF-4 activity was detected in astrocytes, indicating active Wnt signaling in human astrocytes (FIG. 11). IFN-γ markedly reduced this signal by approximately 50% (FIG. 11). These data in conjunction with the TCF-4 ChIP and dominant-negative transfection data indicate that active Wnt signaling is associated with HIV restriction in astrocytes and that IFN-γ overcomes this restriction by reducing the potency of this pathway. This is the first indication that IFN-γ regulates Wnt signaling, which can be harnessed to restrict HIV replication.

### Materials and Methods

Cell Culture: PBMCs were isolated by Ficoll-Hypaque density gradient centrifugation from venous blood collected from healthy laboratory workers. PBMCs were suspended in RPMI 1640 media (Biowhittaker; Walkersville, Md.) supplemented with 10% heat-inactivated fetal bovine serum (FBS) (Sigma, St Louis, Mo.), 1% penicillin/streptomycin (GIBCO-BRL, Grand Island, N.Y.), 2 mM L-glutamine (GIBCO-BRL, Grand Island, N.Y.), and human rIL-2 (20 U/ml, AIDS Research Reagent and Reference Program, Germantown, Md.). The cells were subsequently stimulated with 1 g/ml soluble anti-CD3 and anti-CD28 antibodies (PharMingen; San Diego, Calif.), as indicated. J1.1 cells were cultured in complete media as indicated above but without IL-2. To induce HIV from the latently infected J1.1 cell line, the cells were stimulated with 100 U/ml of TNFα (R & D systems, Minneapolis, Minn.) for 24 hours. Lithium was purchased from Sigma (St. Louis, Mo.) and filter sterilized.

HIV Infection: PBMCs were infected with either the laboratory-adapted CXCR-4 utilizing HIV IIIB isolate or a CCR-5 utilizing primary isolate 302151 (93US151). Chemokine co-receptor usage was determined using the P4-R5 MAGI system as indicated, (AIDS research and reference reagent program, Germantown, Md.). HIV infection of PBMCs was performed by incubating PBMCs with HIV at 10 ng p24/1×10⁶ cells for 2 h at 37° C. Subsequently, unbound virus was removed by washing the cells at least twice. The cells were then cultured in complete media supplemented with IL-2. In some experiments, lithium chloride (Sigma) was added in various amounts (0-1 mM) post-infection and cells cultured at 37° C. and 5% CO₂. HIV replication was monitored seven days post-infection by harvesting the supernatants, lysing the virions with 10% triton X-100 for 1 hour at 37° C., and measuring HIV p24 by conventional ELISA (AIDS vaccine program; Fredrick, Md.).

Cell viability, apoptosis, and cell turnover assay: Cell viability was monitored using the trypan blue exclusion assay. The level of apoptosis was evaluated by an Annexin V/Propidium Iodide (PI) flow-based assay. This assay was performed according to the manufacturers' instructions (BD Biosciences, Franklin Lakes, N.J.). Cell turnover was evaluated using the Carboxycfluoroscein Succinimidyl Ester (CFSE) dye tracking assay. Briefly, PBMCs were stained with CFSE according to manufacturer's instructions (BD Biosciences, Franklin Lakes, N.J.), stimulated with a-CD3/CD28, or left unstimulated, and LiCl (0-25 mM) was added, where indicated. Dilution of the CSFE dye was monitored at time 0, 48, 72 and 96 hrs treatment. All flow-based assays analyses were performed on a FACSCalibur Flow Cytometer utilizing CELLQuest software (BD, Franklin Lakes, N.J.)

DNA Constructs and Transfection: TOPflash and FOPflash constructs, which consist of four native or mutated TCF/LEF DNA binding sites, respectively, linked to luciferase were purchased from Upstate (Billerica, Mass.). CMV Renilla construct was purchased from Promega (Madison, Wis.). The green fluorescent protein (GFP) construct was purchased from Amaxa (Gaithersburg, Md.). The TCF-4 and β-catenin dominant negative mutant constructs were a gift from Dr. James O'Kelly (UCLA, CA) and Dr. Jane B. Trepel (Center for Cancer Research, National institutes of Health, Bethesda, Md.), respectively. Both constructs are described previously PBMCs were transfected using the human T cell nucleofector kit, as recommended by the manufacturer (Amaxa, Gaithersburg, Md.). To measure basal Wnt activity, 5×10⁶ PBMCs were transfected with 3 mg of either TOPflash, FOPflash, or GFP along with 0.03 mg CMV Renilla construct. Luciferase reporter activity was measured by the dual luciferase assay, according to the manufacturer's protocol (Promega, Madison Wis.), using a Monolight 2010 luminometer (BD bioscience) and normalizing to Renilla relative light units values. Loss of function studies was performed by inhibiting endogenous TCF-4 or β-catenin activity by transfecting 3-5×10⁶ PBMCs with their respective dominant negative construct at 5-10 mg of DNA per experimental condition. The total DNA amount was consistent within each experiment. Twenty four hrs post-transfection, PBMCs were infected with HIV and HIV levels measured by conventional ELISA on day seven post-infection.

Western Blot: β-catenin expression was evaluated by conventional western blot. Cell lysates containing 5 μg of protein were resolved on SDS-PAGE and transferred to nitrocellulose membranes (Hy-bond C Super; Amersham). The membrane was incubated with mouse anti-human active-β-catenin (ABC) (US Biological, Massachusetts) at 0.5 μg/ml for 2 hours then with 1:5000 dilution of horse radish peroxidase (HRP)-conjugated goat anti-mouse IgG antibody (Imgenex, San Diego, Calif.). The signals were revealed by enhanced chemiluminescence western blotting (Amersham) and visualized by autoradiography. Subsequently, the membranes were stripped, blocked and blotted with GAPDH antibody as a control for equal amounts of proteins in all samples tested.

Statistical Analysis: The data was analyzed using GraphPad Instat 3 software (San Diego, Calif.). Based on data distribution, either paired or unpaired tests were used. P values <0.05 are considered as significant.

Isolation of HFA and Cell Culture. The astroglioma cell line U87MG was obtained from the NIH AIDS Research and Reference Reagents Program (Germantown, Md.). Cells were cultured in Dulbecco's modified Eagle's medium (Gibco Invitrogen, Carlsbad, Calif.) supplemented with 10% fetal bovine serum (Sigma, St. Louis, Mo.) and 2% penicillin-streptomycin (Gibco Invitrogen) and used in these experiments at approximately 80% confluence. HFA were purified from second-trimester aborted fetuses. First, mechanically dissociated fetal brain tissue (Advanced Bioscience Resources, Alameda, Calif.) was cultured in Dulbecco's modified Eagle's medium (Gibco Invitrogen) supplemented with 10% fetal bovine serum, 2% penicillin-streptomycin, and amphotericin B (Fungizone; Gibco Invitrogen). At each passage, the cells were incubated with 1× trypsin-EDTA (Gibco Invitrogen) for 5 min at 37° C., and the microglia, which remained attached, were discarded while the detached cells, astrocytes, were used for the next passage, cells from which were cultured for 2 weeks. This was repeated for three passages until the cultures were greater than 95% positive for glial fibrillary acidic protein, an astrocyte-specific marker, and less γ than 2% positive for CD68, a microglial marker, as measured by flow cytometry.

IFN-γ Treatment and HIV Infection. Astrocytes were treated with 100 ng/ml of IFN-γ (BD Pharmingen, San Jose, Calif.) or left untreated for 24 h followed by HIV infection and maintenance of IFN-γ postinfection. For infection experiments, astrocytes at 70 to 80% confluence were incubated with HIVBAL (NIH AIDS Research and Reference Reagents Program, Germantown, Md.) at 10 ng p24/1×10⁶ cells for 24 h and then washed three times. In some experiments, cells were trypsinized after HIV infection to further ensure removal of bound virus prior to culturing. HIV infection was monitored at day 7 postinfection by measuring p24 levels by conventional enzyme-linked immunosorbent assay (ELISA) (National Cancer Institute, Frederick, Md.).

Immunofluorescence Staining and Flow Cytometric Analysis. To assess the level of purity of HFA cultures, 1×10⁶ cells were detached with EDTA and incubated with 5% human serum and 1% bovine serum albumin for 30 min at room temperature. Cells were washed, permeabilized, fixed according to a standard protocol (Caltag, Burlingame, Calif.), and stained with mouse anti-glial fibrillary acidic protein (BD Pharmingen) conjugated to allophycocyanin or fluorescein isothiocyanate, mouse anti-CD68-phycoerythrin, mouse anti-microtubule-associated protein 2-allophycocyanin, and mouse antinestin-fluorescein isothiocyanate antibodies (BD Pharmingen). Fluorescence was evaluated with a FACSCalibur flow cytometer using FACSCalibur software (Becton Dickinson, Franklin Lakes, N.J.). Only cultures that were >95% pure were used.

DNA Isolation and Real-time PCR. DNA was isolated using Trizol, as recommended by the manufacturer (Invitrogen, Carlsbad, Calif.) from IFN-γ-stimulated or untreated U87MG cells and primary HFA and quantitated by conventional light absorption at 260/280 nm using a spectrophotometer. For each real-time PCR, 100 ng of DNA was amplified using a PCR mix containing 0.05 μm each of forward and reverse primers, 1×SYBR green, 1.5 mM MgCl2, 0.25 mM deoxynucleoside triphosphates, and 0.02 μl Taq polymerase (Applied Biosystems, Foster City, Calif.). The primers used were R/U5 to amplify early reverse transcripts, R/5NC to amplify late reverse transcripts (24), SK145/150 to amplify gag/pol DNA (4b), or TAR1/TAR2 to amplify the bp 463 to 615 TAR HIV genomic sequence (accession no. K03455). Primer sequences for TAR1/TAR2 are TGGTTAGACCAGATCTGAGCC and TGACTAAAAGGGTCTGAG GGA, respectively. For quantitative SYBR green real-time PCR, the J1.1 cell line (NIH AIDS Research and Reference Reagents Program, Germantown, Md.), which contains one proviral copy of HIV DNA per cell, was used as the HIV DNA standard at 2.5×10¹, 2.5×10², 2.5×10³, 2.5×10⁴, 2.5×10⁵, and 2.5×10⁶ copies per reaction. The amplification reaction consisted of an initial step at 94° C. for 10 min and then 40 cycles at 94° C. for 30 seconds, 60° C. for 30 seconds, and 72° C. for 30 seconds, with a final extension step at 72° C. for 10 min.

ChIP Assay. The formaldehyde cross-linking and chromatin immunoprecipitation (ChIP) assays of astrocytes were performed as described by de La Fuente et al (2000) Briefly, 5×10⁶ cells per immunoprecipitation (IP) were used. TCF-4 antibody was purchased from Upstate (Charlottesville, Va.). Conditions for the ChIP included amplification of the desired target, as indicated, after formaldehyde fixation of the samples but prior to any IP step, IP without the addition of an antibody, or IP using TCF-4 antibody. Pulled-down products underwent realtime PCR amplification using the SYBR green PCR kit (Applied Biosystems, Foster City, Calif.) and primer sequences and PCR conditions as indicated above.

Transfection. Primary human astrocytes and U87MG cells were transiently transfected using nucleofection, as recommended by the manufacturer (Amaxa, Gaithersburg, Md.). Briefly, 5×10⁶ cells were transfected with 10 μg of TOPflash, consisting of native TCF/LEF binding sites linked to a luciferase reporter vector (Upstate, Billerica, Mass.), 1 ng Renilla construct (Promega, Madison, Wis.), or green fluorescent protein (GFP) (pMaxGFP) construct as a control for transfection efficiency and to equalize the total amount of DNA used per transfection condition. Transfected cells were then left untreated or treated with IFN-γ, and luciferase reporter activity was evaluated 24 h later by the dual luciferase assay, as recommended by the manufacturer (Promega, Madison, Wis.). Luciferase values were normalized to Renilla activity. In some experiments, 3×10⁶ to 5×10⁶ cells were transfected with a TCF-4 dominant-negative mutant (James O'Kelly, UCLA, CA), which is a specific inhibitor of the β-catenin/TCF-4 complex (49), or with pMaxGFP, prior to infection with HIV, and p24 levels were measured at day 7 postinfection. Statistical analysis. Descriptive statistics and graphical analysis were used. Nonparametric tests, such as the Wilcoxon rank sum test, were used as appropriate. GraphPad Instat software was used for data analysis.

TABLE 1
A list of putative TCF-4 binding sites

| | | |
|---|---|---|
| Gene, bp, | | |
| strand | code | Oligo sequence: Bio-5′-3′ |

Controls

| | | |
|---|---|---|
| c-Myc | TCF4_c-Myc_wt_pos | TCCCGTCTAGCACCTTTGATTTCTCCCAAA |
| | TCF4_c-Myc_wt_neg | TTTGGGAGAAATCAAAGGTGCTAGACGGGA |
| | TCF4_c-Myc_mut_pos | TCCCGTCTAGCACCTTTGgcTTCTCCCAAA |
| | TCF4_c-Myc_mut_pos | TTTGGGAGAAGCCAAAGGTGCTAGACGGGA |

HIV-1

| | | |
|---|---|---|
| −324: LTR | LAV(111-150)neg | GTACTAGCTTGTAGCACCATcCAAAGGTCAGTGGATATCT |
| | LAV(111-150)pos | AGATATCCACTGACCTTTGGATGGTGCTACAAGCTAGTAC |
| −275: LTR | LAV(171-210)neg | GGTGTAACAAGCTGGTGTTCTCTCCTTTGTTGGCTTCTTC |
| | LAV(171-210)pos | GAAGAAGCCAACAAAGGAGAGAACACCAGCTTGTTACACC |
| −104: LTR | LAV(336-365)neg | CCCAGCGGAAAGTCCcTTGTAGCAAGCTCG |
| | LAV(336-365)pos | CGAGCTTGCTACAAGGGACTTTCCGCTGGG |
| −14: TAR | LAV(441-480)neg | TCAGATCTGGTCTAACCAGAGAGACCCAGTACAGGCAAAA |
| | LAV(441-480)pos | TTTTGCCTGTACTGGGTCTCTCTGGTTAGACCAGATCTGA |
| +6: TAR | LAV(461-500)neg | TAGCCAGAGAGCTCCCAGGCTCAGATCTGGTCTAACCAGA |
| | LAV(461-500)pos | TCTGGTTAGACCAGATCTGAGCCTGGGAGCTCTCTGGCTA |
| +26: TAR | LAV(481-520)neg | TAAGCAGTGGGTTCCCTAGTTAGCCAGAGAGCTCCCAGGC |
| | LAV(481-520)pos | GCCTGGGAGCTCTCTGGCTAACTAGGGAACCCACTGCTTA |

TABLE 2
EXPECTED FRAGMENT SIZE OF WNT MRNAS, AS
AMPLIFIED BY RT-PCR (SUPERARRARY BIOSCIENCE
CORPORATION, FREDERICK, MD).

| Gene | Size (bp) |
|---|---|
| WNT2 | 150 |
| WNT3 | 158 |
| WNT4 | 172 |
| WNT6 | 93 |
| WNT11 | 202 |
| WNT16 | 171 |
| WNT5A | 163 |
| WNT7A | 206 |
| WNT14 | 175 |
| WNT2B | 206 |
| WNT15 | 165 |
| GAPDH | 168 |

TABLE 3
A list of commercially available primary and
secondary antibodies for target proteins.

| Target protein | Source of 1⁰ Ab/2⁰ Ab |
|---|---|
| GFAP | Pig/Donkey |
| β-catenin | Mouse/Donkey |
| p24 | Goat/Donkey |
| CD68 | Goat/Donkey |
| β-catenin | Mouse/Donkey |
| p24 | Rabbit/Donkey |
| CDS | Rat/Donkey |
| β-catenin | Mouse/Donkey |
| p24 | Rabbit/Donkey |
| GFAP | Pig/Donkey |
| p24 | Goat/Donkey |
| β-catenin | Mouse/Donkey |
| IFNγ | Rabbit/Donkey |

TABLE 4
COST OF FDA-APPROVED ANTIHIV DRUGS PER MONTH
COST PER MONTH (estimates)

| | |
|---|---|
| Zidovudine | $180.48 |
| Didanosine | $220.83 |
| Aptivus | $1117.5 |
| Combivir | $752.64 |
| Epivir 300 mg | $347.11 |
| Epzicom | $813.55 |
| Fuzeon | $2315.4 |
| Hivid | $273.00 |
| Trizivir | $1164.3 |
| Truvada | $867.99 |

### DOCUMENTS

Chung, E. J. et al Blood 100, 982 (Aug. 1, 2002).
Xie, D. et al., Cancer Res 64, 1987 (Mar. 15, 2004).

We claim:

1. A method of reducing HIV replication in target cells, the method comprising:
(a) selecting a signaling component in a Wnt signaling pathway;
(b) modulating the signaling component to activate Wnt signaling pathway in the target cells; and
(c) determining the reduction of HIV replication in the target cells.

2. The method of claim 1, wherein the Wnt signaling pathway is activated by lithium chloride (LiCl).

3. The method of claim 1, wherein the signaling component is selected from a group of kinases consisting of GSK-30, β-catenin, and TCF-4.

4. The method of claim 1, wherein the target cells are peripheral blood mononuclear cells.

5. The method of claim 1, wherein the target cells are astrocytes.

6. A method for activating the Wnt signaling pathway in blood cells by
(a) selecting an agent that activates the Wnt pathway; and
(b) administering to the blood cells.

7. The method of claim 1, wherein the target cells are T-cells.

8. A method of reducing HIV replication in a target cell, the method comprising:
(a) selecting a signaling component in a Wnt signaling pathway;
(b) modulating the signaling component by administering an agent to the cell;
(c) activating the Wnt signaling pathway; and
(d) reducing the HIV replication in the target cell.

9. A method to reduce HIV replication in a host target cell, the method comprising:
(a) selecting a signaling component in an intrinsic signaling pathway; and
(b) modulating the signaling component to inhibit HIV replication in the host target cell.
